(19) United States
(12) Patent Application Publication
(10) Pub. No.: US 2015/0038869 A1
(43) Pub. Date: Feb. 5, 2015

(54) SYSTEMS AND METHODS FOR THE PHYSIOLOGICAL ASSESSMENT OF BRAIN HEALTH AND THE REMOTE QUALITY CONTROL OF EEG SYSTEMS

(71) Applicants: Adam J. Simon, Yardley, PA (US); David M. Devilbiss, Madison, WI (US)

(72) Inventors: Adam J. Simon, Yardley, PA (US); David M. Devilbiss, Madison, WI (US)

(73) Assignee: Cerora, Inc., Bethlehem, PA (US)

(21) Appl. No.: 14/233,292

(22) PCT Filed: Jul. 13, 2012
(86) PCT No.: PCT/US2012/046723

§ 371 Date: Aug. 6, 2014

Related U.S. Application Data
(60) Provisional application No. 61/508,638, filed on Jul. 16, 2011.

(51) Int. Cl. A61B 5/0484; A61B 5/00; G01D 18/00; A61B 5/048

(52) U.S. Cl. CPC A61B 5/0484; A61B 5/048; A61B 5/4088; G01D 18/002
USPC 600/544; 702/85

(57) ABSTRACT

A system for calibrating and/or verifying system performance of a remote portable EEG system having at least one EEG sensor. Embodiments of the invention can provide various reference signals to calibrate and quality control the remote performance of the data acquisition EEG system. In addition a calibration cable connects a reference signal source to the EEG sensors to enable remote calibration and quality control assessment. Further, a diagnostic biomarker is included to assess the state or function of a subject's brain and enables the classification, prognosis, diagnosis, monitoring of treatment, or response to therapy applied to the brain by measuring any one of a list of candidate features extracted from a given cognitive or sensory task, and measuring changes in the EEG feature and task combination over time, among multiple states, or compared to a normative database.

### CROSS REFERENCE TO RELATED APPLICATIONS

This application claims priority under 35 U.S.C. §119(e) to U.S. Provisional Application No. 61/508,638, filed on Jul. 16, 2011, which is incorporated herein by reference in its entirety.

### BACKGROUND OF THE INVENTION

1. Technical Field

The invention relates to diagnosis and analysis of brain health through the use of activated tasks and stimuli in a system to dynamically assess one's brain state and function.

2. Description of Related Art

Normal functioning of the brain and central nervous system is critical to a healthy, enjoyable and productive life. Disorders of the brain and central nervous system are among the most dreaded of diseases. Many neurological disorders such as stroke, Alzheimer's disease, and Parkinson's disease are insidious and progressive, becoming more common with increasing age. Others such as schizophrenia, depression, multiple sclerosis and epilepsy arise at younger age and can persist and progress throughout an individual's lifetime. Sudden catastrophic damage to the nervous system, such as brain trauma, infections and intoxications can also affect any individual of any age at any time.

Most nervous system dysfunction arises from complex interactions between an individual's genotype, environment and personal habits and thus often presents in highly personalized ways. However, despite the emerging importance of preventative health care, convenient means for objectively assessing the health of one's own nervous system have not been widely available. Therefore, new ways to monitor the health status of the brain and nervous system are needed for normal health surveillance, early diagnosis of dysfunction, tracking of disease progression and the discovery and optimization of treatments and new therapies.

Unlike cardiovascular and metabolic disorders, where personalized health monitoring biomarkers such as blood pressure, cholesterol, and blood glucose have long become household terms, no such convenient biomarkers of brain and nervous system health exist. Quantitative neurophysiological assessment approaches such as positron emission tomography (PET), functional magnetic resonance imaging (fMRI) and neuropsychiatric or cognition testing involve significant operator expertise, inpatient or clinic-based testing and significant time and expense. One potential technique that may be adapted to serve a broader role as a facile biomarker of nervous system function is electroencephalography (EEG), which measures the brain's ability to generate and transmit electrical signals. However, formal lab-based EEG approaches typically require significant operator training, cumbersome equipment, and are used primarily to test for epilepsy.

Alternate and innovative biomarker approaches are needed to provide quantitative measurements of personal brain health that could greatly improve the prevention, diagnosis and treatment of neurological and psychiatric disorders. Unique tests and biomarkers of Alzheimer's disease, along with the ability to remotely calibrate and quality control EEG systems is a pressing need.

### BRIEF SUMMARY OF THE INVENTION

The systems and methods of the present invention relate to calibrating and conducting quality control assessments of EEG systems remotely without a trained technician involved and using the calibrated EEG systems to assess the brain health of a subject by measuring EEG responses to a variety of stimuli and processing the responses to develop indicators of personalized physiological brain health. In particular, a system for calibrating and/or verifying system performance of a remote portable EEG system having at least one EEG sensor is provided that has at least one ground electrode, a signal generator producing at least one channel of reference signals, a wired cable assembly that connects the signal generator output to the at least one EEG sensor and ground electrode, and a programmed processor that generates test reference signals and collects responses generated by the EEG sensor to the test reference signals to confirm system calibration and and/or verify system performance of the remote portable EEG system.

In exemplary embodiments, the signal generator includes a sound card assembled into a microprocessor based device. The signal generator generates reference signals including linear combinations of sine, square, and triangle waves of varying frequency and amplitude. The reference signals also may include a short circuit between the reference signal and ground enabling a short circuit noise assessment. Generally, the programmed processor is programmed with software algorithms that enable the coordination of the generation of reference signals and the data collection of such reference signals for automated system verification and validation.

In further exemplary embodiments, the wired cable assembly contains a voltage divider to diminish test reference signal amplitudes to physiologically relevant levels. In one embodiment, the wired cable assembly contains a removable voltage divider to diminish test reference signal amplitudes to physiological levels when in place or to calibrate reference signal amplitudes on an individual device by device level when removed from the wired cable assembly.

The scope of the invention also includes systems and methods for assessing the state or function of a subject's brain. In such embodiments, a portable EEG sensing device acquires a subject's EEG signal data during cognitive or sensory testing and a feature extraction system processes the subject's EEG signal data to establish a noninvasive biomarker in the brain that enables the classification, prognosis, diagnosis, monitoring of treatment, or response to therapy applied to the brain by measuring an extracted EEG feature or EEG features from a measured EEG signal when conducting a predetermined cognitive or sensory task. The feature extraction system may also measure changes in the extracted EEG feature or EEG features over time, among multiple states, or compared to a normative database.

In exemplary embodiments, the feature extraction system establishes a biomarker by assessing each block of EEG signal data from the subject to create a list of features, variables or metrics extracted from each block of EEG signal data collected during an individual cognitive task, the list of features, variables or metrics including at least one of: relative and absolute delta, theta, alpha, beta and gamma sub-bands, the theta/beta ratio, the delta/alpha ratio, the (theta+delta)/(alpha+beta) ratio, the relative power in a sliding two Hz window starting at 4 Hz and going to 60 Hz, the 1-2.5 Hz power, the 2.5-4 Hz power, the peak or mode frequency in the power spectral density distribution, the median frequency in the power spectral density, the mean or average (1^{st} moment) frequency of the power spectral density, the standard deviation of the mean frequency (square root of the variance or 2^{nd} moment of the distribution), the skewness or 3^{rd} moment of the power spectral density, and the kurtosis or 4^{th} moment of the power spectral density. The EEG feature or EEG features extracted by the feature extraction system may further include the relative power spectral density within the 18<=f<=20 Hz frequency range of a measured EEG signal when conducting the predetermined cognitive or sensory task, the feature extraction system further establishing a cut-point between 0 and 100 percent for the relative power spectral density across the 18-20 Hz range. In a particular embodiment of the invention, the non-invasive biomarker comprises statistically significant EEG features of Alzheimer's Disease based on the p-value of a statistical significance test applied to the subject.

In further exemplary embodiments, the predetermined cognitive or sensory task further includes at least one of a resting state Eyes Open task, a resting state Eyes Closed task, a Fixation task, a CogState Attention task, a CogState Identification task, a CogState One Card Learning task, a CogState One Card Back task, a Paced Arithmetic Serial Auditory Task (PASAT), a King-Devick Opthalmologic task, a neuro-opthalmologic task, a monaural beat auditory stimulation task, a binaural beat auditory stimulation task, an isochronic tone auditory stimulation task, a photic stimulation task, an ImPACT task, a SCAT2 task, a BESS task, a vestibular eye tracking task, or a dynamic motor tracking task.

In additional exemplary embodiments of the invention, the feature extraction system further diagnoses a disease state of a brain and nervous system of a subject by acquiring EEG signal data of the subject during a resting state task using the portable EEG sensing device, measuring the relative power spectral density of the subject's EEG signal data in a designated frequency sub-band, applying a predetermined cut-point to dichotomize the power spectral density results into one or more biomarker states or classes, and determining which biomarker class a subject belongs to based on the subject's individual power spectral density measurement relative to the predetermined cut-point.

In other exemplary embodiments of the invention, the feature extraction system extracts an EEG feature or EEG features by applying discrete or continuous wavelet transformation analysis to the subject's EEG signal data to identify statistically meaningful features.

### BRIEF DESCRIPTION OF THE DRAWINGS

Embodiments of the invention can be better understood with reference to the following drawings, of which:

FIG. 1 is a schematic diagram illustrating the remote calibration and quality control system of the invention.

FIG. 2 is a schematic diagram illustrating a two channel calibration cable for remote quality control of an EEG system.

FIG. 3 is a schematic diagram illustrating a one channel calibration cable for remote quality control of an EEG system.

FIG. 4 is a graph showing the frequency response of a EEG system including six Fast Fourier Transformed (FFT) Power Spectral Density (PSD) traces calculated from a raw EEG signal collected with a NIST traceable signal generator at from 5 to 30 Hz in 5 Hz steps.

FIG. 5 is a graph showing the amplitude response of an EEG system as the amplitude is reduced by 50% steps at 15 Hz showing a well behaved 4-fold reduction in power across a large amplitude range from 80 μV down to 1.25 μV after stepping down thru a 10⁴ voltage divider as illustrated in FIG. 2 or FIG. 3.

FIG. 6 is a table of Signal to Noise Ratios (SNR) in either the time domain of voltage or frequency domain of frequency for four different experiments with input sine wave of 15 Hz from a NIST traceable function generator.

FIG. 7 is a two trace graph comparing an expensive NuAmps 10-20 reference EEG system by Compumedics to the inexpensive and portable Cerora MindScope system. The data were collected simultaneously but show good agreement in frequency and amplitude response.

FIG. 8A is a graph showing an EEG signal with several artifacts which are being detected by pre-processing artifact detection software.

FIG. 8B is a table showing the detection efficiency of the pre-processing artifact detection software algorithms.

FIG. 9A is a 3 dimensional Power Spectral Density plot over time of a noise signal with an inset of the time averaged power spectral density.

FIG. 9B is a 3 dimensional Power Spectral Density plot over time of a linear combination of four equal amplitude sine waves constructed in silico with an inset of the time averaged power spectral density.

FIG. 9C is a 3 dimensional Power Spectral Density plot over time of a linear combination of four unequal amplitude sine waves constructed in silico with an inset of the time averaged power spectral density.

FIG. 9D is a table showing the power in the spectral sub-bands of seven artificially constructed signals, providing verification and validation of the spectral analysis code used in the present invention.

FIG. 10 is a table showing the demographics of the participants in the Palm Drive Pilot Alzheimer's disease study.

FIG. 11 is a table listing the clinical protocol of tasks that the Palm Drive Pilot study participants experienced while EEG data was collected using the system and methods of the present invention.

FIG. 12 is a graph showing a two-second interval of a resting Eyes Open (EO) EEG signal recorded from an Alzheimer's disease participant in the pilot study.

FIG. 13 is a graph of the relative power spectral density (PSD) of the full two minute block of resting EO EEG data shown in part in FIG. 11.

FIG. 14A is a graph comparing the relative theta spectral sub-band power in each of the N=13 Control (CTL) subjects relative to the N=10 Alzheimer's disease subjects in the pilot study, showing an increase in relative theta power in Alzheimer's disease subjects.

FIG. 14B is a graph comparing the relative beta spectral sub-band power in each of the N=13 Control (CTL) subjects relative to the N=10 Alzheimer's disease subjects in the pilot study, showing a decrease in relative beta power in Alzheimer's disease subjects.

FIG. 15A is a graph comparing the mean frequency in Hertz (Hz) across the entire 1-30 Hz PSD in each of the N=13 Control (CTL) subjects relative to the N=10 Alzheimer's disease subjects in the pilot study, showing an overall decrease in mean frequency in Alzheimer's disease subjects.

FIG. 15B is a graph comparing the relative power in the unique biomarker signature comprised of the relative power in the 18-20 Hz portion of the beta sub-band in each of the N=13 Control (CTL) subjects relative to the N=10 Alzheimer's disease subjects in the pilot study, showing an overall decrease in relative 18-20 Hz power in Alzheimer's disease subjects.

FIG. 16 is 2 by 2 diagnostic table showing the clinical performance of the relative 18-20 Hz power biomarker using a 0.27 cut-point to classify those who are control versus those with mild Alzheimer's disease. The sensitivity, specificity, Positive Predictive Value and Negative Predictive Value are calculated to the bottom and right of the 2 by 2 data table. Receiver Operator Characteristic (ROC) curve analysis shows an area under the curve of 0.85 in JMP software.

FIG. 17 is a table listing possible tasks to include in a clinical protocol that sports concussion athletes and mild traumatic brain injury patients could be assessed with while EEG data was collected using the system and methods of the present invention.

FIG. 18 is an example of a raw EEG signal of a subject (Subject 11) before (top) and after (bottom) artifact detection.

FIG. 19 is a diagram showing the discrete wavelet transformation decomposition scheme with 5 levels of decomposition, where D₁-D₅ and A₅ represent the signal.

FIG. 20 is a series of traces showing the discrete wavelet transformation decomposition of an individual subject's EEG signal (top trace) into the various component signals D₁ (2^{nd} from top), D₂ (3^{rd} from top), D₃ (4^{th} from top), D₄ (5th from top), D₅ (6^{th} from top) and A₅ (bottom).

FIG. 21 is a diagram showing the discrete wavelet transform decision tree analysis results for resting states only, where x₁ is the standard deviation of the {D₄}, corresponding to the θ frequency sub-band, of the second Eyes Open state (EO4), and x₂ is the mean power value of the {D₂}, corresponding to the β sub-band, for the second Eyes Open state (EO4).

FIG. 22 is a diagram showing the discrete wavelet transform decision tree analysis results for active states only, where x₁ is the minimum value of {D₄}, corresponding to the θ sub-band, of auditory binaural beat stimulation at Δ=12 Hz (AS2), x₂ is the maximum value of {D₃}, corresponding to the α sub-band, of auditory binaural beat stimulation at Δ=6 Hz (AS1), and x₃ is the skewness of the {D₃}, corresponding to the a sub-band, of the CogState One Card Back task (CG4).

FIG. 23 is a diagram showing the discrete wavelet transform decision tree analysis results for all states, where x₁ is the skewness {D₅}, corresponding to the upper 8 band, of the fourth Eyes-Closed state (EC7), x₂ is the mean power value of {D₂}, corresponding to the β band, for PASAT 2.0 (s) interval task, and x₃ is the mean power value of the {D₂}, corresponding to the β band, of the first Eyes-Open state (EO2).

FIG. 24 is a diagram showing the continuous wavelet transform decision tree analysis results for resting states only, where x is the absolute mean power of wavelet scales in the scale range 13-26, corresponding to θ frequency sub-band, during the Eyes Open EO4 task.

FIG. 25 is a screenshot of the output from a successful quality control procedure which includes diminishing amplitude and changing frequency output from the sound card hardwired to the headset.

### DETAILED DESCRIPTION OF THE INVENTION

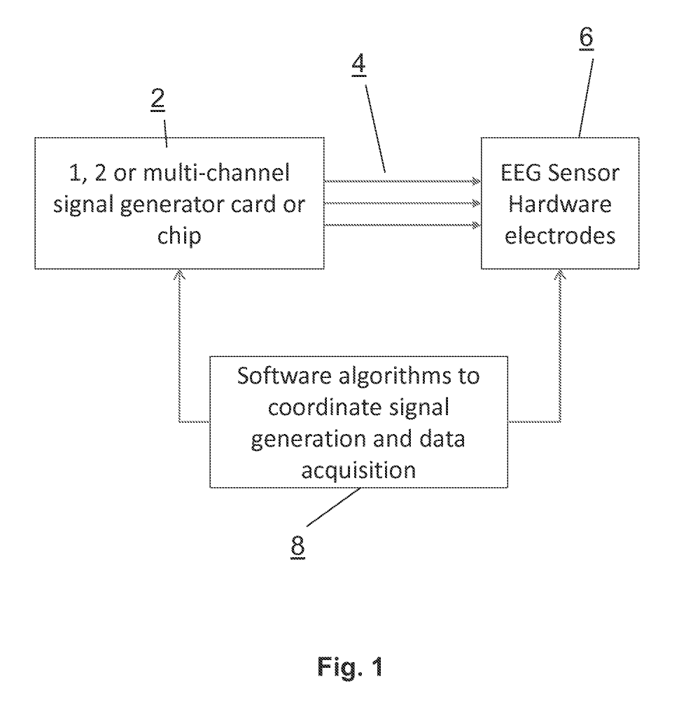

The invention will be described in detail below with reference to FIGS. 1-25. Those skilled in the art will appreciate that the description given herein with respect to those figures is for exemplary purposes only and is not intended in any way to limit the scope of the invention. All questions regarding the scope of the invention may be resolved by referring to the appended claims.

### Definitions

By “electrode to the scalp” we mean to include, without limitation, those electrodes requiring gel, dry electrode sensors, contactless sensors and any other means of measuring the electrical potential or apparent electrical induced potential by electromagnetic means.

By “monitor the brain and nervous system” we mean to include, without limitation, surveillance of normal health and aging, the early detection and monitoring of brain dysfunction, monitoring of brain injury and recovery, monitoring disease onset, progression and response to therapy, for the discovery and optimization of treatment and drug therapies, including without limitation, monitoring investigational compounds and registered pharmaceutical agents, as well as the monitoring of illegal substances and their presence or influence on an individual while driving, playing sports, or engaged in other regulated behaviors.

A “medical therapy” as used herein is intended to encompass any form of therapy with potential medical effect, including, without limitation, any pharmaceutical agent or treatment, compounds, biologics, medical device therapy, exercise, biofeedback or combinations thereof.

By “EEG data” we mean to include without limitation the raw time series of voltage as a function of time, any spectral properties determined after Fourier transformation, any nonlinear properties after non-linear analysis, any wavelet properties, any summary biometric variables and any combinations thereof.

A “sensory and cognitive challenge” as used herein is intended to encompass any form of sensory stimuli (to the five senses), cognitive challenges (to the mind), and other challenges (such as a respiratory CO2 challenge, virtual reality balance challenge, hammer to knee reflex challenge).

A “sensory and cognitive challenge state” as used herein is intended to encompass any state of the brain and nervous system during the exposure to the sensory and cognitive challenge.

An “electronic system” as used herein is intended to encompass, without limitation, hardware, software, firmware, analog circuits, DC-coupled or AC-coupled circuits, digital circuits, FPGA, ASICS, visual displays, audio transducers, temperature transducers, olfactory and odor generators, or any combination of the above.

By “spectral bands” we mean without limitation the generally accepted definitions in the standard literature conventions such that the bands of the PSD are often separated into the Delta band (f<4 Hz), the Theta band (4<f<7 Hz), the Alpha band (8<f<12 Hz), the Beta band (12<f<30 Hz), and the Gamma band (30<f<100 Hz). The exact boundaries of these bands are subject to some interpretation and are not considered hard and fast to all practitioners in the field. These are also called sub-bands by some practitioners.

By “calibrating” we mean the process of putting known inputs into the system and adjusting internal gain, offset or other adjustable parameters in order to bring the system to a quantitative state of reproducibility.

By “conducting quality control” we mean conducting assessments of the system with known input signals and verifying that the output of the system is as expected. Moreover, verifying the output to known input reference signals constitutes a form of quality control which assures that the system was in good working order either before or just after a block of data was collected on a human subject.

By “biomarker” we mean an objective measure of a biological or physiological function or process.

By “biomarker features or metrics” we mean a variable, biomarker, metric or feature which characterizes some aspect of the raw underlying time series data. These terms are equivalent for a biomarker as an objective measure and can be used interchangeably.

By “non-invasively” we mean lacking the need to penetrate the skin or tissue of a human subject.

By “diagnosis” we mean any one of the multiple intended use of a diagnostic including to classify subjects in categorical groups, to aid in the diagnosis when used with other additional information, to screen at a high level where no a priori reason exists, to be used as a prognostic marker, to be used as a disease or injury progression marker, to be used as a treatment response marker or even as a treatment monitoring endpoint.

By “statistical predictive model” we mean the method of analysis where input variables and factors are assembled and analyzed according to predescribed rules or functions to either classify a subject into a category (state A, state B or state C) or to predict an continuous outcome variable, such as the probability to progress to a state B from a state A or the likelihood of disease in any one individual given their input factors or variables. Any of the methods of the book The Elements of Statistical Learning: Data Mining, Inference, and Prediction (Second Edition) by Trevor Hastie, Robert Tibshirani and Jerome Friedman (2009), are non-limiting examples of predictive statistical models.

By “multiple states” we mean any one of the non-limiting variety of brain states that can be assessed, such as before versus after administration of a therapy, before versus after a putative injury, before versus after a putative disease state.

By “diagnostic EEG feature” we mean any one individual variable or derived characteristic of the many possible nominal, ordinal or continuous variables that can be derived from the raw EEG data which was stored or analyzed as voltage as function of time raw data. These can be uni-variate in nature or multi-variate, assembled from two or more individual features or characteristics used in combination. These features can be used in any statistical predictive model or decision tree, either logistic or regressive in nature, as an input variable or input factor.

### Systems and Methods for Calibrating and Conducting Quality Control of EEG Systems Remotely Without a Trained Technician

The systems and methods of the present invention comprise cables and reference signals which can easily be delivered locally to calibrate an EEG hardware/software system remotely without formal training or additional equipment. It is often necessary to insure the integrity and good calibration of electronic equipment controlled by software. Often trained operators and engineers conduct detailed and extensive calibration procedures with scientific instruments traceable to a reference standard like a National Institutes of Standards and Testing (NIST) traceable standard. Certificates of Analysis often link a local calibration to a known reference standard. The same needs to be true for portable and remotely used functional EEG systems and methods, similar to those disclosed in PCT patent application PCT/US2010/038560 to the present assignee. However, if a portable or disposable system is moved outside of a clinic or hospital setting, it is equally important for mobile health devices to remain calibrated in electrical and mechanical properties. Unfortunately, often problems emerge like an intermittent contact or a complete disruption of an electrical conductor or contact. Often electrical components can fail and an operator or subject may not know that everything is not working.

A solution to this problem includes a remote calibration and quality control system which is a part of the hardware/software system to collect the remote EEG signals. Typically, a remote EEG data collection device includes a microprocessor with a wired or wireless data communication protocol like USB or Bluetooth which interfaces to the EEG sensor data stream in one direction with a high bandwidth connection to a communication network, such as a mobile cellular telecommunications network, Wi-Fi internet network, or satellite network connection in the other direction. In many common instances, the microprocessor will be part of a portable device such as laptop personal computer, net book, Bluetooth enabled smart or feature phone, iPod touch, Android device or other dedicated hardwire device, as non-limiting examples. In each case, a signal generator or sound card is typically available within the device. This is true for many of the available microprocessor based consumer based devices; in particular this is true for laptop PCs, net books, smart or feature phones, the iPod touch and Android devices.

As illustrated in FIG. 1, the systems and methods of the present remote calibration and quality control invention include (i) a signal generator card or chip 2, often including a sound card or other audio signal generator, to generate test or reference signals, (ii) a cable 4 to hardwire the sound card output (typically from a headphone jack with a 2.5 mm or 3.5 mm male connector) to the electrodes 6 of the remote and portable EEG hardware, and (iii) custom software 8 built into the data acquisition software program that is able initiate reference signal generation from the signal generator or sound card in a specified fashion to calibrate the frequency response and amplitude response of the EEG data acquisition system. If there is more than one channel of EEG data to record, then a multi-channel calibration signal can calibrate the phase relationship between any two channels of the data acquisition information streams as well. Typically, a sound card or sound chip outputs stereo signals with two channels of output, although monophonic sound cards or chips with one channel or 5.1 or 7.1 surround-sound cards or chips can equally be used within the system and methods of the present invention.

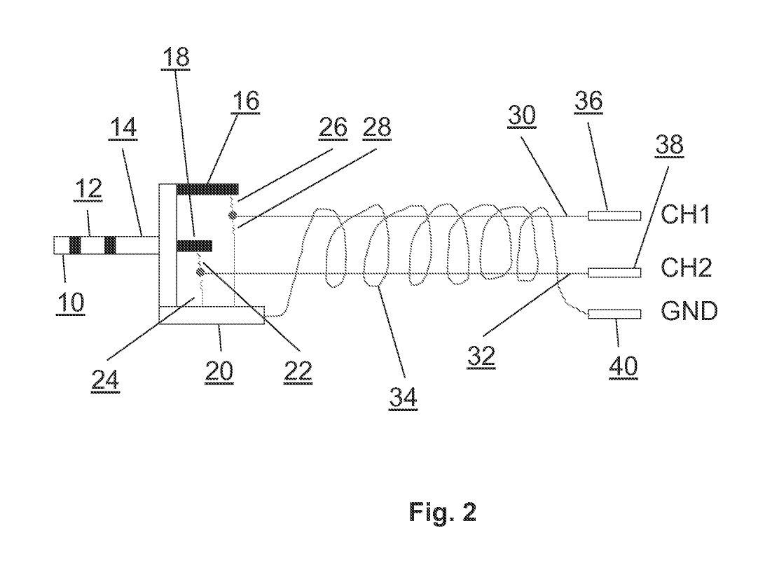

An example of a stereo two-channel calibration cable is shown in FIG. 2. As illustrated, the male jack pin has a first conductor (e.g. L left channel) 10 which is passed thru to pin 18, while a second signal conductor (e.g. R right channel) 12 is passed thru to pin 16. The ground electrode 14 is attached to the shield 20 of the cable assembly. Wired into the cable assembly is a voltage divider consisting of upper resistor 26 and lower resistor 28 for channel 1 while upper resistor 22 and lower resistor 24 make up the voltage divider for channel 2. Wire 30 carries the 10³ to 10⁴ voltage reduced signal for channel 1 to connector 36 which is attached to an electrode on the EEG recording device by an alligator clip or other mechanically and electrically stable means. Moreover, wire 32 carries the voltage reduced channel 2 signal to connector 38. The ground of the jack pin is connected via wire 34 and is wrapped around the two signal wires to shield the signals and is attached to the ground and/or reference electrode on the EEG recording device by connector 40.

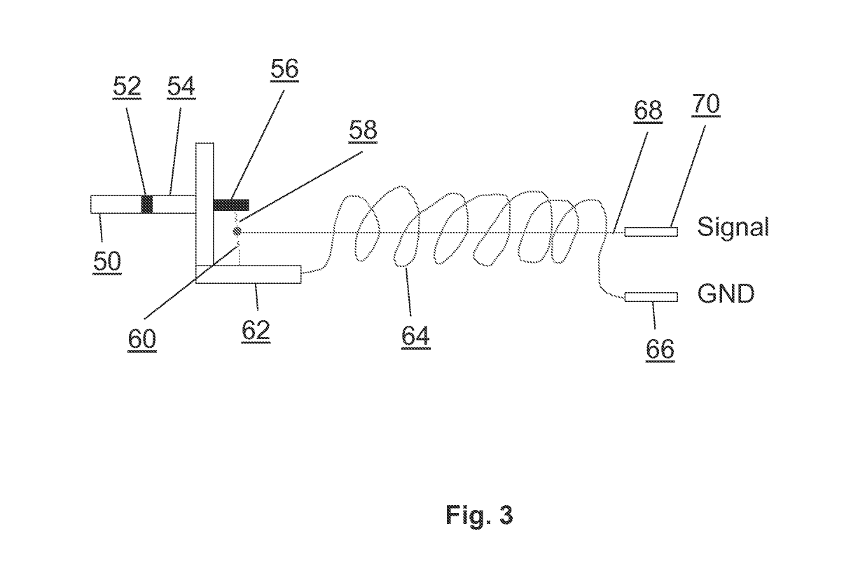

Another embodiment of a calibration system in accordance with the present invention would be a single channel cable assembly as shown in FIG. 3. In this case, pin connector 50 passes thru to pin 56, while insulator 52 separates ground conductor 54 which is electrically continuous with cable shield 62 and shielding wire or signal wrap 64. A voltage divider is created between upper resistor 58 and lower resistor 60 to step down the reference signals by 10³ to 10⁴, although it may only be necessary to step down 10² or as much as 10⁵. The voltage divided signal is passed along signal wire 68 to connector 70. The ground shield wire or foil 64 is connected via connector 66 to the ground or reference electrodes in the data acquisition system.

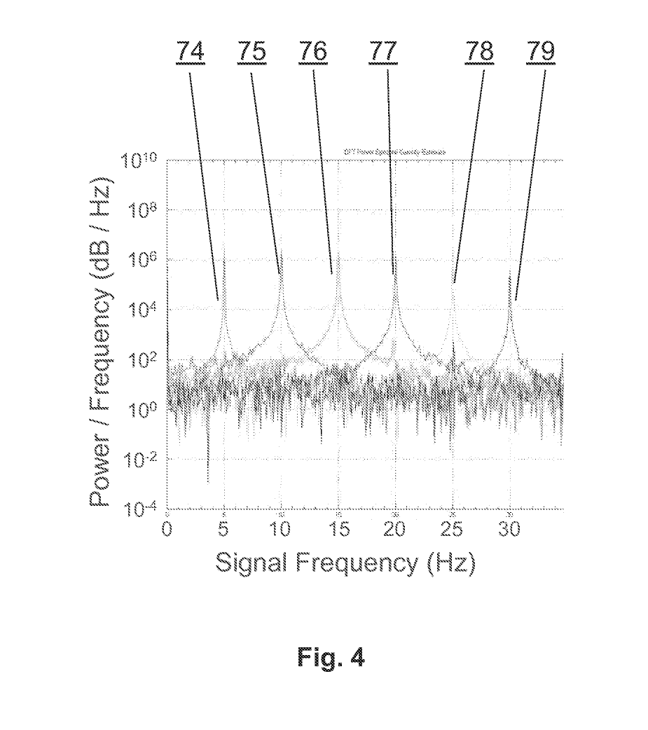

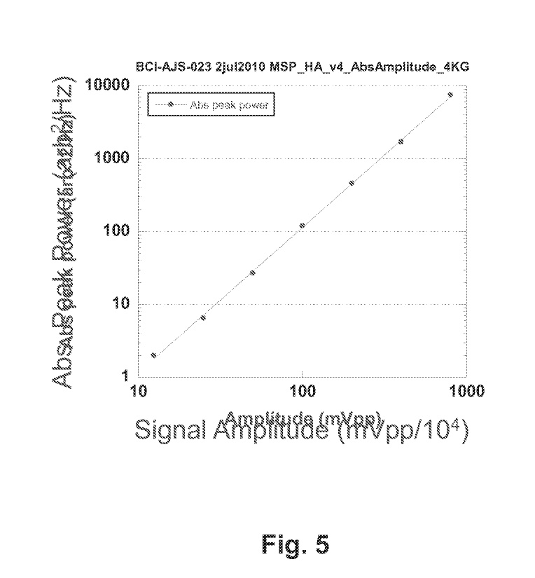

An example of a frequency response output can be seen after Fourier Transform in FIG. 4. Measured power spectral density (PSD) traces at 5 Hz (74), 10 Hz (75), 15 Hz (76), 20 Hz (77), 25 Hz (78) and 30 Hz (79) can be seen aligning well with expectation. Alternatively, an amplitude scan can be automated by the signal generating software and signals at a fixed or mixed frequency collected at varying amplitude (see FIG. 5). One sees a two-fold reduction in input signal amplitude along the x-axis corresponding to an expected 25% reduction in power. This power law model fit tracks very well demonstrating excellent amplitude response.

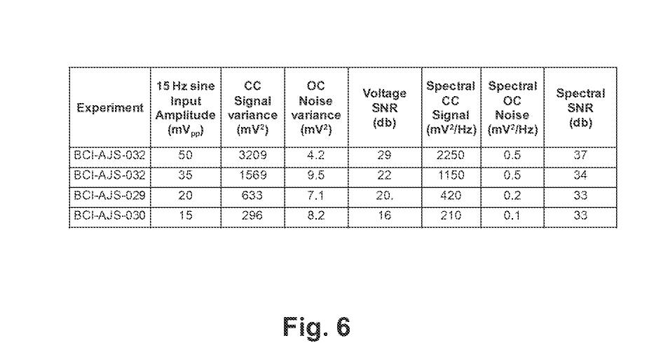

Depending on the switching capabilities of the signal generating card, one can possibly conduct a signal to noise ratio in real time by shorting signal and ground outputs and recording noise levels compared to physiologically referenced levels. Such a test could produce an output table as shown in FIG. 6 where the SNR is shown in both the time and frequency domains.

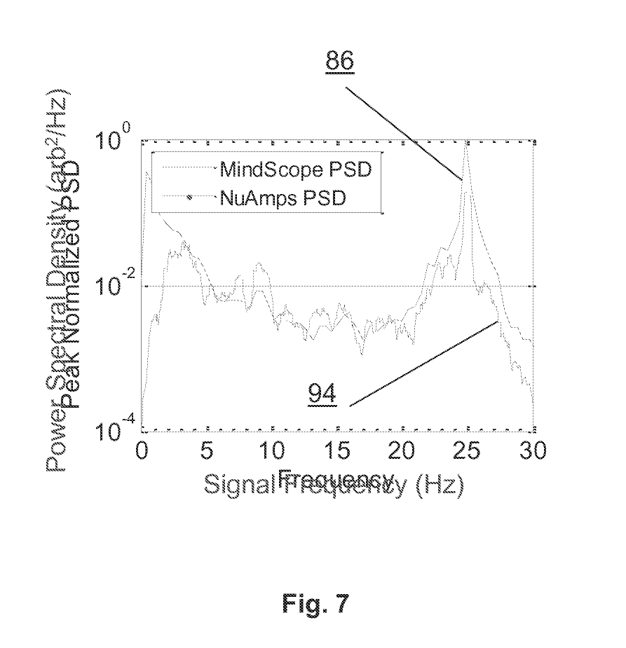

The system and methods of the present invention show comparable frequency response and PSD as an expensive reference system as shown in FIG. 7. FIG. 7 is a two trace graph comparing an expensive NuAmps 10-20 reference EEG system (signal 86) by Compumedics to the inexpensive and portable Cerora MindScope system (signal 94). The data were collected simultaneously but show good agreement in frequency and amplitude response. However, because the two systems were connected in parallel, there was interaction between the two systems which lead to an artifact at 25 Hz. Nonetheless, it was observed equivalently in both systems.

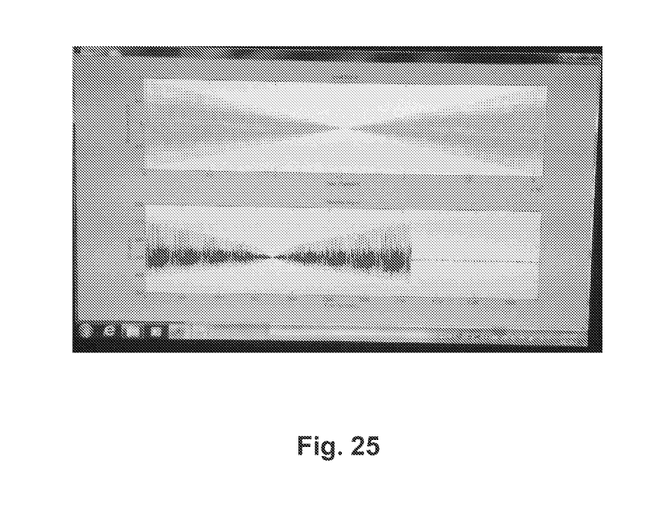

The output from a ramped 30 second passage of test reference signals can be observed in FIG. 25, showing the reference output above relative to the measured signal from the system below. Here both the frequency and the amplitude are changing in time to assess both the calibration and quality of the system across a range of control parameters, such as frequency and amplitude.

### Systems and Methods to Verify and Validate Analytic Software Modules

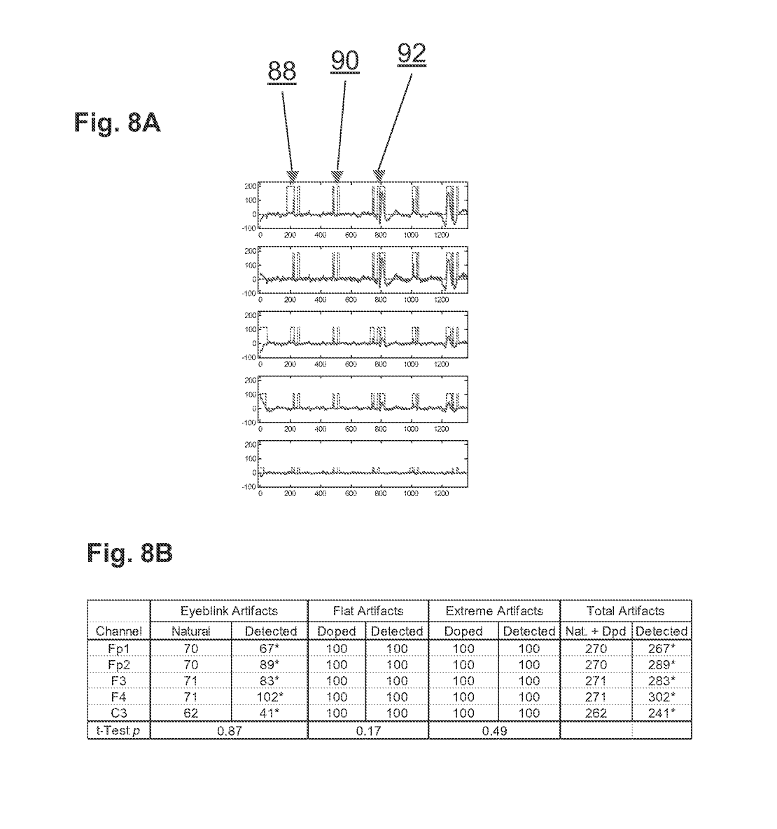

The test signals of the present invention can be used to verify and validate analytic software modules written to achieve explicit purposes. Preferred embodiments enable the verification and validation of pre-processing artifact detection algorithms. In particular, if the signal generator chip has the capability to stream digitally synthesized artifacts or stored artifact signals, then the pre-processing analysis algorithms can be verified and validated for use. An example of this can be seen in FIG. 8A where various artifacts 88, 90 and 92 were flagged and excluded from the epochs of artifact free EEG data. In FIG. 8B, a table of naturally occurring or synthetically doped in silico artifacts versus those detected by the pre-processing software was constructed showing an excellent efficiency at detecting known or doped artifacts of the eye blink, flat (no variation over many samples), or extreme (saturated signal) types.

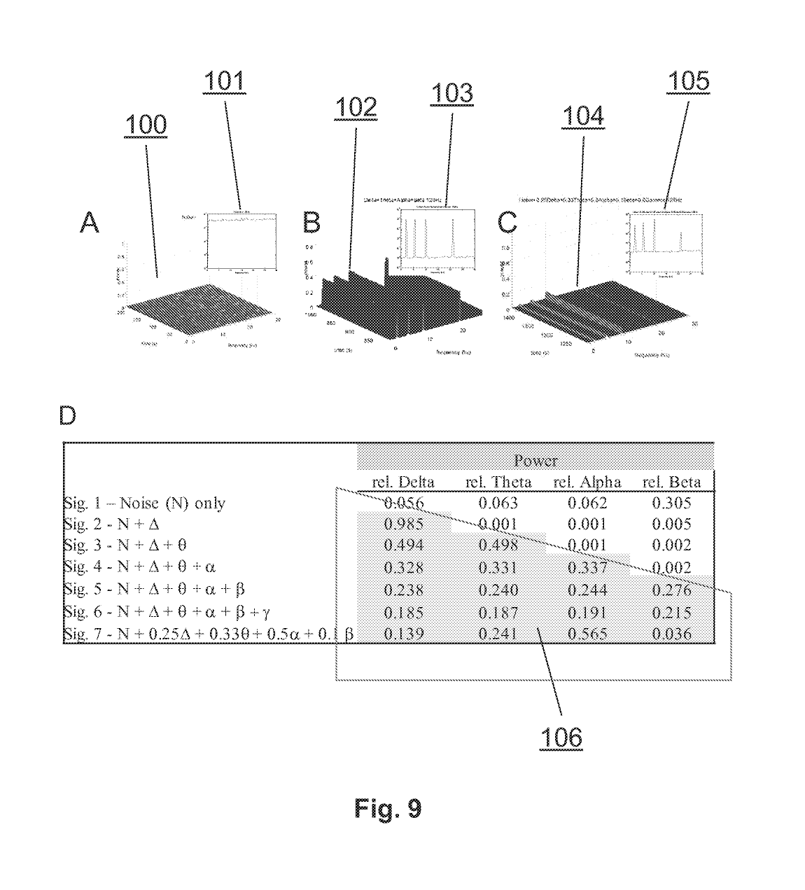

Moreover, synthetically created signals in the signal generator card can be constructed with varying linear combinations of amplitudes and frequencies to verify and validate that the data acquisition system is performing as expected and is within calibration specification before additional human clinical data is gathered and/or stored for analysis. This ability provides a very important quality control and assurance to the human clinical data remotely collected by a patient or subject without a trained operator or technician present to confirm in an automated fashion, proper and calibrated collection of the EEG data. FIG. 9A shows white noise in both a 3-dimensional 100 and 2 dimensional time average 101 PSD plot. When four sine waves of equal amplitude are combined into a single artificial test waveform in FIG. 9B, they are detected as equal amplitude in both a 3-dimensional 102 and 2-dimensional time average 103 PSD plot. Finally, if the linear combination of reference signals does not have equal amplitude, as shown in FIG. 9C, then the 3-dimensional 104 or 2-dimensional time averaged 105 PSD can be shown to have the proper relative power in each of the intended sub-bands, as documented in FIG. 9D in the lower triangle of sub-band power values 106. Moreover, by allowing convenient remote monitoring using the present invention, the remote calibration and quality control and assurance activities can be automated and can be undertaken much less expensively than the present status.

Biomarkers and Methods to Diagnose Brain Disease (e.g. Alzheimer's Disease)

The system and methods of the present invention also relate to the ability to non-invasively measure with a lightweight, portable and user-friendly system, EEG-derived biomarker features or metrics extracted from the raw time series traces of EEG data. These features can then be placed into a summary data table alongside other available data and information to enable statistical predictive models using as many co-variates as possible that can be constructed during the statistical analysis phase. Moreover, multi-variate methods such as linear discriminant analysis, tree based methods such as Random Forest method, and other multi-variate statistical methods can be conducted to create multi-variate composite biomarkers that can demonstrate better analytical and clinical performance to screen, classify, diagnose, prognose, monitor brain or disease progression, or monitor drug response. All of these methods fall into the general term diagnose as alternative intended uses of the systems, markers and methods of the present invention.

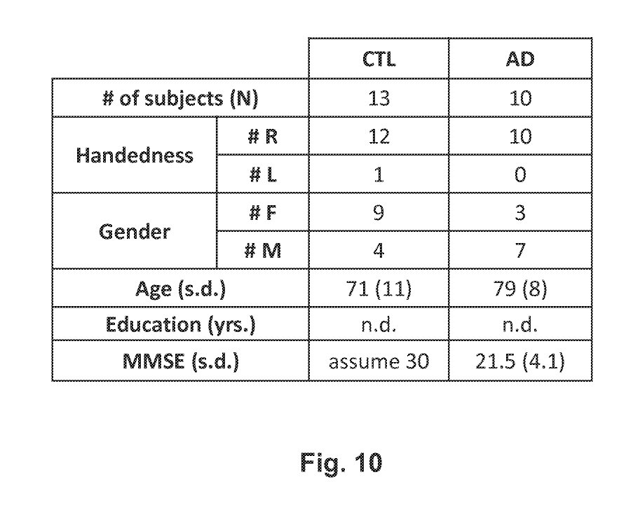

In one exemplary embodiment of the present invention, subjects would get enrolled after either (i) IRB approval as an Investigation Device or (ii) after FDA 510(k) clearance or (iii) after FDA Pre-market Approval (PMA). Demographic data would be collected on each subject included their handedness, gender, age, education, concomitant medications, blood pressure, diabetes and smoking history, along with any other imaging or biomarker data available to establish either standard of truth or other possible co-variates in the analysis. See FIG. 10 for an example of the collected data.

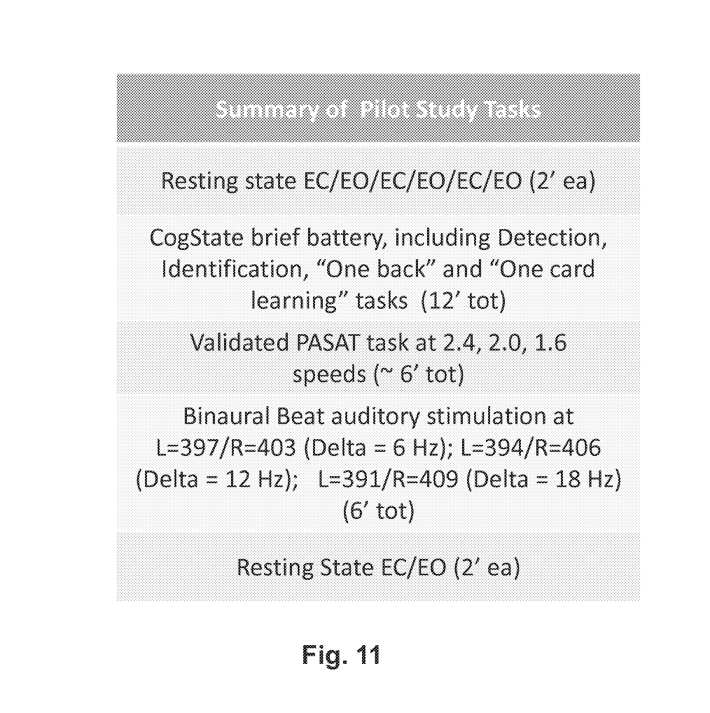

Once ready to conduct the diagnostic procedure using the system and methods of the present invention, a clinical assessment protocol beginning with both resting state Eyes Closed (EC) and resting state Eyes Open (EO) conditions would be initiated (see FIG. 11 for an example). This would alternate for three successive cycles for a total of six blocks of resting state data in one embodiment, or alternatively consist of one, two or four cycles of EC and EO resting states. From there, the computer acquisition system would begin the physiologically focused cognitive or sensory stimulation tasks while recording EEG signals. In one particular embodiment, EEG signals would be recorded while a cognitive or sensory visualization series of tasks were initiated. In one embodiment, the CogState Brief Battery was conducted, including the Detection, Identification, “One Card Back”, and “One Card Learning” tasks for a total of 4 additional blocks of data taking roughly an additional 12 minutes. Other non-limiting tasks would include the ImPACT neurological assessment, the Cantab battery or other visualization tasks or ANAM.

Next, the software would present to the subject an auditory cognitive or sensory task probing the auditory cortex and requiring speech responses. One such embodiment could include the PASAT task starting at the slowest speed of 2.4 seconds between trials, then begin again at the next faster speed of 2.0 seconds between trials, and if the subject agreed, conducted for a third and final time at the 1.6 seconds between trial speeds. Alternatively, a verbal task such as the King-Devick Test developed in ophthalmology could be used to assess speech and visual acuity. After this, the device sound card would be hooked up to iPod like ear-buds or other audio transducer on the subject and would begin to output auditory stimulation to probe the auditory cortex with direct sounds and tones. In one preferred embodiment, a binaural beat frequency would be setup through differentiated left and right ear frequencies. In one particular embodiment, the tones would be centered in a pitch range between 40-400 Hz with differential delta beat frequency varying from 1 to 30 Hz. In a more particular embodiment, a central frequency of 400 Hz would be used with a binaural beat delta frequency of 6 Hz, then 12 Hz, then 18 Hz, each block recording from 15 seconds to two minutes of EEG signals. Other center frequency and beat frequency combinations could be equally contemplated. Alternative auditory stimulations could include monoaural beats and isochronic tones. An opportunity to include photic stimulation of the subject with eye lids closed could be conducted according to the methods of the present invention. The frequency of photic stimulation could be varied from 1 to 2 Hz on the slow side through 30 to 40 Hz on the fast side. The appearance of primary driving frequency signals as well as the presence of first harmonic signals could be monitored and used a biomarker signature to help in the diagnosis protocol.

The existence of either the primary driving frequency or the first harmonic or higher harmonics could be a nominal or ordinal variable output. Moreover, continuous output variables such as the amplitude of the driving frequency peak, first harmonic peak amplitude, or ratio to a resting state comparator could be used as a diagnostic EEG feature. Also possible, the continuous output variable relative or absolute power in the driving frequency or the harmonics could be used as a diagnostic EEG feature. Pain stimuli in the form of a thermal grill or an ice cube to the hand could be implemented to assess the coupling of peripheral circuits to the central nervous system and frontal or other cortical areas. Finally the activation/stimulation battery of cognitive and sensory tasks would end with a resting state EC/EO sequence for a block of data each of duration 2 minutes.

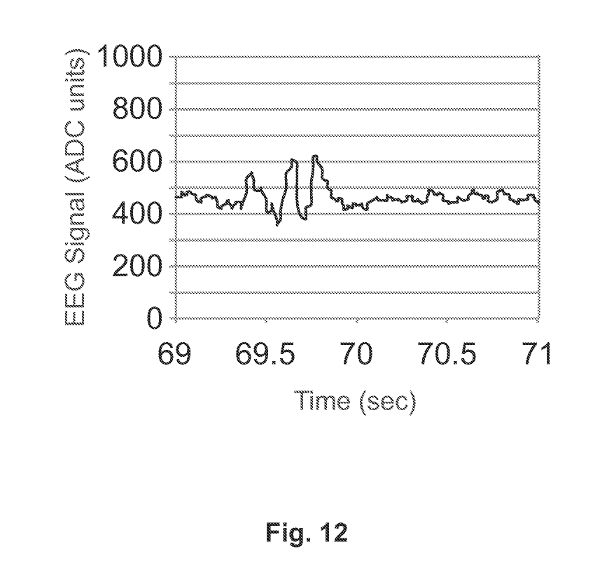

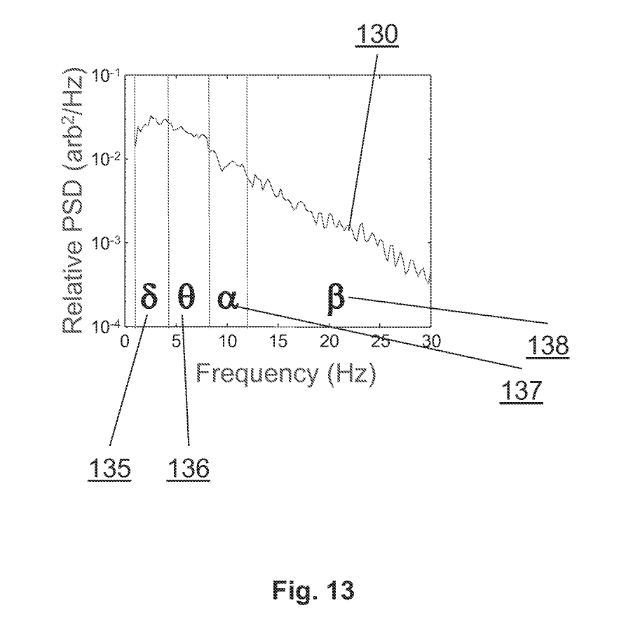

EEG time series would be recorded into the various data blocks as described above. FIG. 12 shows an example two second time series sampled at 128 Samples/sec with 10-bit ADC sample resolution. This could then go through the pre-processing artifact detection algorithms and those epochs that were not flagged as artifact would be Fast Fourier transformed into the frequency domain and either plotted without normalization as an absolute power spectral density or could alternatively be normalized to overall power of unity and represented as the relative power spectral density (PSD). An example relative PSD trace 130 can be seen in FIG. 13, where the various sub-bands have been indicated by the vertical lines on the plot. The slowest frequency sub-band known as delta, typically from 1-4 Hz, can be seen at 135, followed by the theta sub-band from 4-8 Hz at 136, followed by the alpha sub-band from 8-12 Hz at 137, followed by the beta sub-band from 12-30 Hz shown at 138. Not shown on the plot in FIG. 13 is the gamma sub-band from 30-60 Hz because with a sampling frequency of only 128 samples/sec, one can choose to not go all the way up to the Nyquist frequency but more rigorously require at least 4 samples per unit cell. If one uses a 256 samples/sec or 512 samples/sec ADC, then meaningful gamma sub-band information can be ascertained.

It should be noted that the algorithms and “processing means” described herein are preferably implemented in software that runs on a processor of the processing unit (which is presumably part of the portable EEG sensing device).

Once the spectral analysis code has transformed each epoch of artifact free time series EEG data, a feature extraction algorithm can assess each block of transformed data to create a list of features or variables or biomarkers extracted from each block of EEG data conducted during an individual task. This list of variables or metrics can include not only the relative and absolute delta, theta, alpha, beta and gamma sub-bands, but can include literature derived markers such as the theta/beta ratio, the delta/alpha ratio, the (theta+delta)/(alpha+beta) ratio, the relative power in a sliding two Hz window starting at 4 Hz and going to 60 Hz, the 1-2.5 Hz power, the 2.5-4 Hz power, the peak or mode frequency in the PSD distribution, the median frequency in the PSD, the mean or average (1^{st} moment) frequency of the PSD, the standard deviation of the mean frequency (square root of the variance or 2^{nd} moment of the distribution), the skewness or 3^{rd} moment of the PSD, the kurtosis or 4^{th} moment of the PSD. In addition to these spectrally derived metrics or features for each block of EEG data, non-spectral signal analysis could be conducted.

In an exemplary embodiment of the present invention, a non-linear dynamics module would calculate the largest Lyaponov exponent of the block of EEG data, the fractal dimension D of the EEG signal and the entropy S of the EEG signal, as non-limiting non-linear dynamical systems extracted EEG features. In an alternate embodiment, a wavelet transform signal analysis module could be applied to an all artifact free EEG epoch on a block by block basis. This analysis could include both the discrete wavelet transform (DWT) as well as continuous wavelet transform (CWT). More particularly, these advanced signal analysis routines would be applied to blocks of EEG data acquired during either cognitive or sensory stimulation to enhance diagnostic discriminatory power.

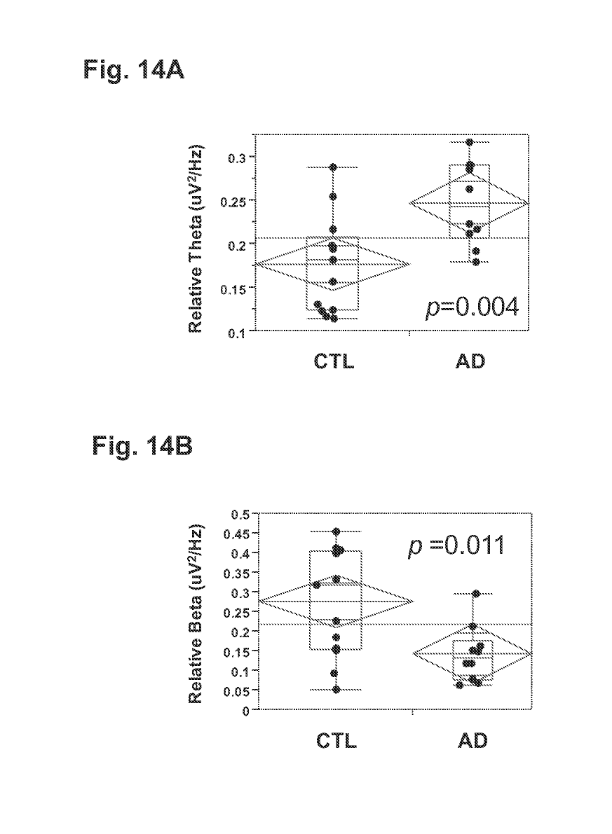

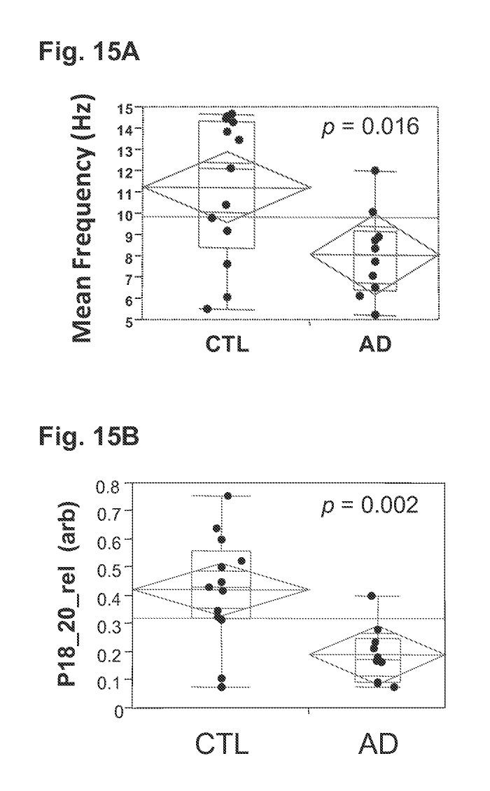

As a non-limiting example, a two group plot of the relative theta power during a resting Eyes Open task between N=10 Alzheimer's subjects and N=13 Control subjects averaged over three blocks of two minutes each can be seen in FIG. 14A. In FIG. 14B, one can visualize the decreased relative beta sub-band in AD relative to CTL, again in resting EO. In both plots, the false positive rate t-Test p-value is shown to be statistically meaningful when not correcting for multiple comparisons. Moreover, as can be seen in FIG. 15A, the mean frequency is meaningfully reduced from approximately 11 Hz in CTL subjects to around 8 Hz in AD subjects, again with a statistically meaningful t-Test p-value.

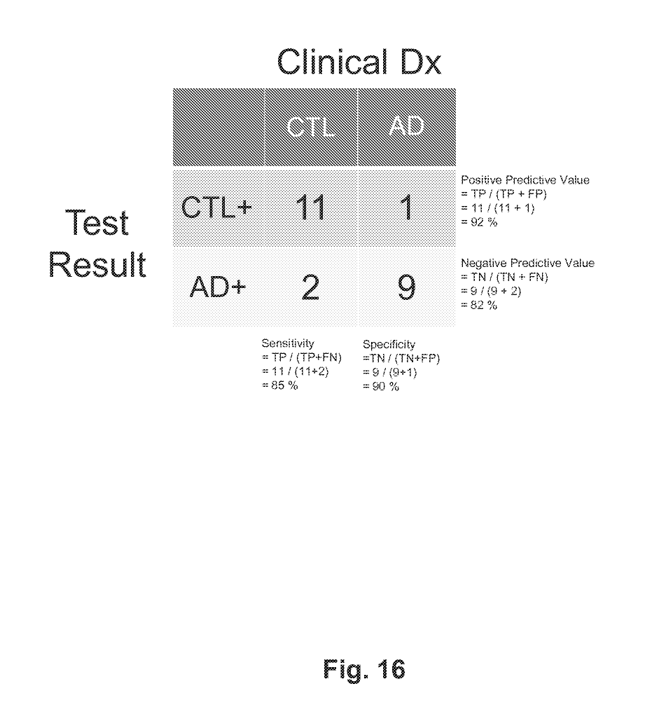

Alternatively, additional features or metrics beyond those described in the literature can be extracted from the artifact free EEG blocks of data. In FIG. 15B, the relative 18-20 Hz window of power averaged over three blocks of resting EO can be seen to have excellent diagnostic discriminatory power. In fact, if one conducts a nominal logistic regression to perform Receiver Operator Characteristic (ROC) curve analysis, an optimal cut-point to dichotomize the groups is observed at relative 18-20 Hz power of 2.7. When pivoting on this cut-point as shown in FIG. 16, the preliminary clinical performance of the relative 18-20 Hz power as a biomarker or classifier can be seen to correctly identify 11 of 13 CTL subjects and 9 of 10 AD subjects, based on a clinical diagnosis as standard of truth. This marker thus has a derived 85% sensitivity with 90% specificity, while the Positive Predictive Value (PPV) is 92% and the Negative Predictive Value is 82%.

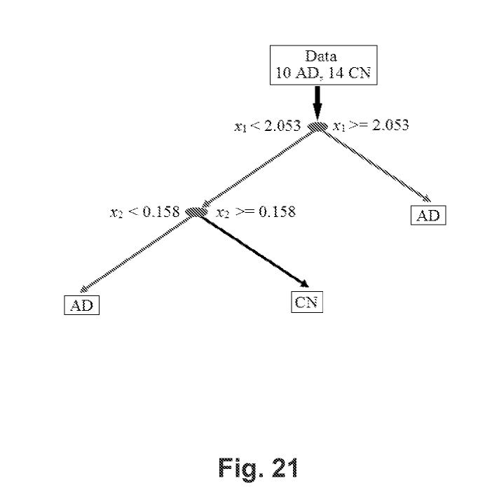

Alternatively, one can conduct either discrete or continuous wavelet transformation analysis of the EEG blocks of data. In a discrete wavelet analysis, as described in Example 14 below, one can see the statistically meaningful results with false positive rate p<0.05 shown in Table 1. More results can be found within FIG. 21 through FIG. 24.

These results indicate that any one of the following extracted EEG feature by task combinations can be used alone or in combination as an input or factor to a statistical predictive model for Alzheimer's disease:
1. mean power D2 during an Eyes Open (EO) task,
2. mean power D3 during an EO task,
3. mean power D4 during an EO task,
4. mean power D5 during an EO task,
5. minimum D2 during an EO task,
6. minimum D3 during an EO task,
7. minimum D5 during an EO task,
8. maximum D2 during an EO task,
9. maximum D4 during an EO task,
10. maximum D5 during an EO task,
11. Standard Deviation (STD) D2 during an EO task,
12. STD D3 during an EO task,
13. STD D4 during an EO task,
14. STD D5 during an EO task,
15. Kurtosis D5 during an EO task,
16. mean power D3 during an Eyes Closed (EC) task,
17. mean power D4 during an EC task,
18. minimum D3 during an EC task,
19. minimum D4 during an EC task,
20. maximum D4 during an EC task,
21. STD D3 during an EC task,
22. STD D4 during an EC task,
23. Skewness D5 during an EC task,
24. Ajlasdfj,
25. minimum D2 during an One Card Learning task,
26. minimum D3 during an One Card Back task,
27.skewness D3 during a One Card Back task,
28.skewness D2 during a D=6 Hz binaural beat auditory stimulation task,
29. minimum value of {D₄} during an auditory binaural beat stimulation at Δ=12 Hz (AS2),
30. maximum value of {D₃} during an auditory binaural beat stimulation at Δ=6 Hz (AS1),
31. skewness of the {D₃} during a CogState One Card Back task (CG4),
32.skewness {D₅} during an EC task,
33. mean power value of {D₂} during a PASAT 2.0 (s) interval task,
34. mean power value of the {D₂} during a EO task, and
35. absolute mean power of wavelet scales in the scale range 13-26 during an EO task.

TABLE 1
Statistically significant task by extracted EEG features from the
Discrete Wavelet Transformation analysis and Kruskal-Wallis test.

| | EC | EO | EC | EO | EC | EO | CG | CG | CG | CG | P | P | AS | AS | AS | EC | EO |
|---|---|---|---|---|---|---|---|---|---|---|---|---|---|---|---|---|---|
| | 1 | 2 | 3 | 4 | 5 | 6 | 1 | 2 | 3 | 4 | 2.4 | 2.0 | 1 | 2 | 3 | 7 | 8 |
| Power Mean | — | .04 | — | .003 | — | .035 | — | — | — | — | — | — | — | — | — | — | — |
| D2 | | | | | | | | | | | | | | | | | |
| Power Mean | — | — | — | .022 | .04 | — | — | — | — | — | — | — | — | — | — | — | — |
| D3 | | | | | | | | | | | | | | | | | |
| Power Mean | — | .08 | — | .0001 | .001 | — | — | — | — | — | — | — | — | — | — | — | .026 |
| D4 | | | | | | | | | | | | | | | | | |
| Power Mean | — | — | — | .0005 | — | .046 | — | — | — | — | — | — | — | — | — | — | — |
| D5 | | | | | | | | | | | | | | | | | |
| Minimum | — | .04 | — | .007 | — | — | — | — | .04 | — | — | — | — | — | — | — | — |
| D2 | | | | | | | | | | | | | | | | | |
| Minimum | — | — | — | .011 | .001 | — | — | — | — | — | — | — | — | — | — | — | — |
| D3 | | | | | | | | | | | | | | | | | |
| Minimum | — | — | — | — | .007 | .022 | — | — | — | — | — | — | — | — | — | — | — |
| D4 | | | | | | | | | | | | | | | | | |
| Minimum | — | — | — | .002 | — | .016 | — | — | — | — | — | — | — | — | — | — | — |
| D5 | | | | | | | | | | | | | | | | | |
| Maximum | — | — | — | .016 | — | .046 | — | — | — | — | — | — | — | — | — | — | — |
| D2 | | | | | | | | | | | | | | | | | |
| Maximum | — | — | — | — | — | — | — | — | — | .0098 | — | — | — | — | — | — | — |
| D3 | | | | | | | | | | | | | | | | | |
| Maximum | — | — | — | .026 | .021 | — | — | — | — | — | — | — | — | — | — | — | — |
| D4 | | | | | | | | | | | | | | | | | |
| Maximum | — | — | — | .04 | — | .016 | — | — | — | — | — | — | — | — | — | — | — |
| D5 | | | | | | | | | | | | | | | | | |
| STD | — | .04 | — | .003 | — | .035 | — | — | — | — | — | — | — | — | — | — | — |
| D2 | | | | | | | | | | | | | | | | | |
| STD | — | — | — | .022 | .047 | — | — | — | — | — | — | — | — | — | — | — | — |
| D3 | | | | | | | | | | | | | | | | | |
| STD | — | .022 | — | .0001 | .001 | — | — | — | — | — | — | — | — | — | — | — | .026 |
| D4 | | | | | | | | | | | | | | | | | |
| STD | — | — | — | .0006 | — | .04 | — | — | — | — | — | — | — | — | — | — | — |
| D5 | | | | | | | | | | | | | | | | | |
| Skewness | — | — | — | — | — | — | — | — | — | — | — | — | .031 | — | — | — | — |
| D2 | | | | | | | | | | | | | | | | | |
| Skewness | — | — | — | — | — | — | — | — | — | .032 | — | — | — | — | — | — | — |
| D3 | | | | | | | | | | | | | | | | | |
| Skewness | — | — | — | — | — | — | — | — | — | — | — | — | — | — | — | — | — |
| D4 | | | | | | | | | | | | | | | | | |
| Skewness | — | — | — | — | — | — | — | — | — | — | — | — | — | — | — | .0005 | — |
| D5 | | | | | | | | | | | | | | | | | |
| Kurtosis | — | — | — | — | — | — | — | — | — | — | — | — | — | — | — | — | — |
| D2 | | | | | | | | | | | | | | | | | |
| Kurtosis | — | — | — | — | — | — | — | — | — | — | — | — | — | — | — | — | — |
| D3 | | | | | | | | | | | | | | | | | |
| Kurtosis | — | — | — | — | — | — | — | — | — | — | — | — | — | — | — | — | — |
| D4 | | | | | | | | | | | | | | | | | |
| Kurtosis | — | — | — | — | — | .03 | — | — | — | — | — | — | — | — | — | — | .02 |
| D5 | | | | | | | | | | | | | | | | | |

In a continuous wavelet analysis, as described in Example 15 below, one can see the statistically meaningful results with false positive rate p<0.05 shown in Table 2. More results can be found within FIG. 21 through FIG. 24.

These results indicate that any one of the following extracted EEG feature by task combinations can be used alone or in combination as an input or factor to a statistical predictive model for Alzheimer's disease:
1. relative power du during an Eyes Open (EO) task,
2. relative power ql during an Eyes Open (EO) task,
3. relative power ql during an Eyes Closed (EC) task,
4. relative power au during an Eyes Open (EO) task,
5. relative power au during an Eyes Closed (EC) task,
6. relative power bl during an Eyes Open (EO) task,
7. relative power bl during an Eyes Closed (EC) task,
8. relative power bu during an Eyes Open (EO) task
9. absolute power δᵤ during an Eyes Open (EO) task,
10. absolute power θₗ during an Eyes Open (EO) task,
11. absolute power θₗ during an Eyes Closed (EC) task,
12. absolute power θᵤ during an Eyes Open (EO) task,
13. absolute power θᵤ during an Eyes Closed (EC) task,
14. absolute power αᵤ during an Eyes Open (EO) task,
15. absolute power αᵤ during an Eyes Closed (EC) task,
16. absolute power βₗ during an Eyes Open (EO) task,
17. absolute power βₗ during an Eyes Closed (EC) task, and
18. absolute power βᵤ during an Eyes Open (EO) task.

TABLE 2
Statistically significant task by extracted EEG features from the
Continuous Wavelet Transformation analysis and Kruskal-Wallis test.

| State | δᵤ | θₗ | θᵤ | αₗ | αᵤ | βₗ | βᵤ |
|---|---|---|---|---|---|---|---|
| | Relative Powers | | | | | | |
| EC1 | 0 | 0 | 0 | 0 | 0 | 0 | 0 |
| EO2 | 0 | 0 | 0 | 0 | 0 | 1, p = 0.046 | 1, p = 0.046 |
| EC3 | 0 | 0 | 0 | 0 | 0 | 0 | 0 |
| EO4 | 1. p = 0.0006 | 1. p = 8.7 × 10⁻⁵ | 0 | 0 | 1. p = 0.007 | 1, p = 0.004 | 1, p = 0.004 |
| EC5 | 0 | 1, p = 0.034 | 0 | 0 | 1, p = 0.025 | 1, p = 0.025 | 0 |
| EO6 | 0 | 1, p = 0.026 | 0 | 0 | 0 | 1, p = 0.035 | 1, p = 0.022 |
| | Absolute Powers | | | | | | |
| EC1 | 0 | 0 | 0 | 0 | 0 | 0 | 0 |
| EO2 | 0 | 0 | 1, p = 0.019 | 0 | 0 | 0 | 0 |
| EC3 | 0 | 0 | 1, p = 0.019 | 0 | 0 | 0 | 0 |
| EO4 | 1, p = 0.0006 | 1, p = 5.3 × 10⁻⁵ | 1, p = 0.007 | 0 | 1, p = 0.026 | 1, p = 0.004 | 1, p = 0.005 |
| EC5 | 0 | 1, p = 0.047 | 1, p = 0.029 | 0 | 1, p = 0.015 | 1, p = 0.018 | 0 |
| EO6 | 1, p = 0.040 | 1, p = 0.016 | 0 | 0 | 0 | 1, p = 0.046 | 1, p = 0.019 |

### EXAMPLES

While the above description contains many specifics, these specifics should not be construed as limitations on the scope of the invention, but merely as exemplifications of the disclosed embodiments. Those skilled in the art will envision many other possible variations that are within the scope of the invention. The following examples will be helpful to enable one skilled in the art to make, use, and practice the present invention.

### Example 1

### Creation of a Remote Calibration Cable Assembly for Remote Quality Control Purposes

Using a soldering iron, resistors, stereo jack pin, wire and alligator clips, a calibration and quality control cable was constructed. The voltage divider consisted of an upper ¼ watt resistor of 100 ohms (Ω) and a lower ¼ watt resistor of 1,000,000 ohms or 1 MΩ to divide the reference signals down by a factor of 10⁴ from 1 volt to 100 μv and 50 mV to 5 μV. These stepped down signals are thus within the typical physiological range of a 1 μV to 100 μV and thus useful for assessment and calibration of EEG systems. If desired, metal film resistors with tighter tolerances could be used.

### Example 2

### Download Human EEG Data and Create a Dummy Brain Setup

Publically available EEG data was downloaded from the UCSD website (http://sccn.ucsd.edu/˜arno/fam2data/publicly_available_EEG_data.html) and stored locally on computers. The various .tar.gz data files were unzipped using BitZipper software and then the .tar files were unpacked into individual files using Astrotite software. Various individual proprietary format, Neuroscan .cnt files (in particular cba1ff01+cba1ff02, cba2ff01+cba2ff02, ega1ff01+ega1ff02, ega2ff01+ega2ff02) were converted into ASCII comma-separated values (CSV) files using the biosig package for Matlab (http://biosig.sourceforge.net/), which were then viewed and loaded into Excel. Sequentially matched EEG data files (based on the UCSD documentation) were concatenated to create samples streams in excess of 65K samples.

An Agilent AT-33220A Function Generator/Arbitrary Waveform Generator (“Arb”) and an Agilent AT-34410A 6.5 digit Digital Multi-Meter (DMM) were rented for use. Each instrument was successfully configured to work with PCs using the Agilent I/O Suite 15.5 libraries and Agilent Connect software with a USB cable (Arb) or Ethernet cable (DMM). EEG data in ASCII format were copied into, and completely filled, one of the 65,536 sample non-volatile buffers available within the Arb hardware using Agilent's “Waveform Editor” software. In total, each of the four concatenated downloaded EEG files (cba1, cba2, ega1, ega2) was stored in the four separate memory buffers on the Arb. These data provided output EEG signal streams of just over 65 seconds, and as a result, the Arb was able to hold 65,536 samples. The UCSD data was recorded at 1,000 Samples/sec according to the documentation. Upon setting the Arb to a frequency of 15.259 mHz (based on 1000 Samples/sec divided by 65,536 samples in the non-volatile buffer=15.258789 sec-1). Waveform amplitude varied, often set between −1.0 V and +1.0 V to yield a voltage resolution of 0.123 millivolts with the 14 bit dynamic range of the Arb. For visual confirmation, output from each of the four non-volatile Arb buffers was observed on a Tektronix digital oscilloscope. The traces appeared to replicate the original downloaded signal shapes as observed in the Waveform Editor software before transfer to the Arb.

### Example 3

### Characterization of the Frequency and Amplitude Response

A one channel calibration and quality control cable was built according to Example 1 as shown in FIG. 3. An Agilent AT-33220A Function Generator/Arbitrary Waveform Generator (“Arb”) and an Agilent AT-34410A 6.5 digit DMM were used. Each instrument was successfully configured to work with laboratory PCs using the Agilent I/O Suite 15.5 libraries and Agilent Connect software with a USB cable (Arb) or Ethernet cable (DMM). Downloaded UCSD EEG data in ASCII format were copied to, and completely filled, one of the 65,536 sample non-volatile buffers available within the Arb hardware using Agilent's “Waveform Editor” software. In total, each of the four concatenated downloaded EEG files (cba1, cba2, ega1, ega2) was stored in the four separate memory buffers on the Arb. These data provided an output EEG signal streams of just over 65 seconds, and as a result of the Arb can hold 65,536 samples. The UCSD data was recorded at 1,000 Samples/sec. Upon setting the Arb to a frequency of 15.259 mHz (based on 1000 Samples/sec divided by 65,536 samples in the non-volatile buffer=15.258789 sec-1), the Arb was able to successfully output publically available EEG data as a dummy brain setup. Output amplitude was set to vary between −1.0 V and +1.0 V to yield a voltage resolution of 0.123 millivolts with the 14 bit dynamic range of the Arb. For visual confirmation, output from each of the four non-volatile Arb buffers was observed on a digital oscilloscope. The traces appeared to replicate the original downloaded signal shapes as observed in the Waveform Editor software before transfer to the Arb.

Additionally, sine wave output from the NIST traceable Arb was hardwired into the EEG headset beginning at 5 Hz and ending at 30 Hz in 5 Hz intervals with modest input amplitude of approximately 25 μV. Each block of independent data was analyzed by pre-processing artifact detection algorithms and then spectral sub-band analysis. The output PSD for each of the six traces can be seen in FIG. 4. As expected, the pure sine waves exhibit excellent spectral peak widths. Furthermore, the frequency of the reference sine wave was fixed at 15 Hz and the input sine wave amplitude to the voltage divider was reduced from 800 mVₚₚ to 12.5 mVₚₚ in a 2 fold serial reduction (e.g. 800, 400, 200, 100, 50, 25, 12.5). After the 10⁴ voltage divider, the input voltage amplitudes to the EEG sensor were 80, 40, 20, 10, 5, 2.5, and 1.25 μVₚₚ, covering well the physiological range. The results of the study can be seen in FIG. 5 where a two-fold reduction in amplitude leads to a 4 fold reduction in power as expected. The linearity of the response looks excellent.

### Example 4

### Assessment of the Open Circuit Signal to Noise Ratio (SNR)

While experiments were conducted under closed circuit conditions as well as under both open circuit and short circuit conditions to assess the signal to noise ratio of the MindScope hardware and recording system. There are primarily two types of noise: short circuit noise when the differential input to the differential operational amplifier are shorted together and open circuit noise due to intermittent pickup of spurious signals when there is no signal presented to the sensor. Relevant literature suggested open circuit noise levels are larger than short circuit noise levels so we began our investigation with open circuit noise assessments in the headsets compared to hardwired signals from the Arb. The literature also suggested that signals more than three standard deviations are more than 99% probably meaningfully different than noise (assuming Gaussian noise). Thus, SNR greater than 10*log (3²/1²)=9.5 db represent real signals with p<0.01. A proposed threshold criterion of 20 db is thus highly conservative. Multiple experiments were conducted to determine the SNR from the data comparing open circuit to close circuit conditions, confirming the reports in the literature.

The SNR data were analyzed both in the voltage-time domain as well as spectral domain. In each case, the log transformed ratio of signal to noise was calculated to determine the SNR in decibels (db). In addition to time-voltage domain SNR measurements,

The average spectral power was measured around 0.1 μV² equivalents averaging across two measurements. This experiment was conducted with multiple trials within each of N=2 separate days. SNR in decibels (db) is defined as ten times the log base 10 ratio of the signal squared divided by the noise squared, where the values are root-mean squared (rms), centered at 15 Hz with a bandwidth from zero to 30 Hz (P. Horowitz and W. Hill, The Art of Electronics, 2^{nd} Edition, Cambridge University Press: 1989, p 434.) The data summarized both in the time domain before transformation (RMS) and after transformation (Spectral) are shown in FIG. 6. Thus, we find that for pure 15 Hz sine wave signals, we calculate a SNR=10 log (210/0.1)=33 db in the spectrally transformed space. In the un-transformed time domain, input signals above 20 mV before the voltage divider or 2 μV input to the EEG sensor will have the necessary 20 db SNR.

### Example 5

### Show Equivalence to a Reference System

Four data files were recorded from signals produced by the Agilent 33220A 20 MHZ Function/Arb Waveform Generator (Arb) simultaneously on the EEG system of the present invention and a Compumedics system (Neuroscan NuAmps amplifier and gel-based electrodes). The Arb was limited to four non-volatile memory buffers for storing UCSD downloaded EEG human data so analysis was limited to these four UCSD EEG data traces (CDA1_{—}1, CDA2_{—}2, EGA1_{—}3, EGA2_{—}4). Compumedics Neuroscan SCAN 4.5 analysis software was successfully installed on laboratory computers. The 1000 Samples/sec NuAmps data files were imported into SCAN 4.5 software in the Compumedics .CNT file format. They were then transformed into “Epoch” files of approximately 1, 2, or 4 seconds in duration and contained 1024, 2048, or 4096 samples. Once broken into epochs, the data were Fast Fourier Transformed yielding either amplitude (μV) or power (μV²) measures and plotted from 0 to 30 Hz. Sensitivity of the PSD was assessed by examining all three epoch lengths (1024, 2048, and 4096 sample lengths) indicating no major deviations in sensitivity were observed. Individual power spectra were exported as ASCII data files for direct comparison between power spectra of Compumedics and the MindScope system. A comparison of the overall relative power spectra (peak normalized) revealed agreement (FIG. 7). However, spectral deviation at the low end delta sub-band was observed.

Additionally, both systems identify an artifactual spectral peak around 25 Hz as a function of output from the Arb. This artifact was seen throughout the experiments conducted with the Arb. As such, the response of the two systems was very comparable with the exception of the frequency response below 3 Hz.

One issue was revealed during the data analysis. There was an apparent interaction between the NuAmps and MindScope systems, due to the periodic injection of electrical current by the EEG headset to test the signal quality of the electrode contact to the human subject. As illustrated in FIG. 7, this periodic injection dramatically affected the PSD observed, but did so equivalently in both the NuAmps and MindScope data.

### Example 6

### Verification and Validation of the Pre-Processing Artifact Detection Algorithms

Pre-processing artifact detection provides a standardized series of detection routines, but additionally permits the user to select from these routines. Artifact detection and removal is critical to EEG signal processing to maximize the accuracy and precision of spectral estimates as well as other measurements used to determine cognitive or sensory state-dependent changes. The developed artifact detection routines assess the EEG for invalid data in the following manner:
1) Determination of samples acquired during periods of time when the EEG signal was poor. Poor Signal occurs during intervals that the headset is not placed upon or properly seated on the head. This is disclosed by a value reported by the EEG wireless headset as a value ranging from 0 to 200. We have determined that signals acquired with a Poor Signal value greater than 26 are not precise enough to use for effective analysis. (defined in the BCI_ParameterFile as params.Artifact.minSignalStrength=26).
2) Determination of Flat Segments (samples acquired during periods of no frequency information—DC only). Flat Segments occur when Bluetooth™ communication to the EEG wireless headset is lost or other conditions such as electromagnetic interference render the signal unusable, including saturation of the amplification circuitry to the limits of the input power supply voltage. We have determined that Flat Segments longer than 100 milliseconds produce significant deviations in spectral estimation. (defined in BCI_ParameterFile as params.Artifact.minFlatSigLength=0.1).
3) Determination of Excessive Signals (samples that exceed three standard deviations of the signal mean). Excessive Signal segments occur during eye blinks, interference of cardiac electrical activity (heart beat), or non-physiological electrical noise including movement of the EEG dry electrode or electromagnetic interference. (defined in BCI_ParameterFile as params.Artifact.maxSignalSTDmultiplier=3).
4) Determination of Excessive Δv/Δt Segments (series of samples that exceed a predetermined instantaneous frequency). These Excessive Δv/Δt Segments occur as a result of non-physiological electrical noise including movement of the EEG dry electrode or electromagnetic interference. We have determined that a change of 1.5 standard deviations from the signal mean over 3 samples is sufficient to detect these non-physiological signals. (defined in BCI_ParameterFile as params.Artifact. dvValMultiplier=0.5 and params.Artifact.MaxDT=3).

The performance of the artifact detection software module was measured to provide quality control and assurance benchmarks. Five separate signals from UCSD data files CBA1ff01 were extracted, down-sampled to 128 Hz, band passed filtered (0.5-50 Hz), and formatted for use. These signals were analyzed visually for known artifacts and eye blinks were counted manually while scanning the data file. No other major artifact was observed. To test each aspect of the artifact detection algorithm, each signal was incrementally seeded with 100 artifacts. Synthetic artifact segments were generated at sub-threshold and super-threshold values that contained: 1) flat signal (i.e. representing dropped signal or amplifier/ADC saturation) or 2) extreme values (i.e. representing electrical noise or other non-physiological signal). Under generic and non-optimized settings (values reported above for each detector parameter), our artifact detection algorithm initially detected 342 of 344 artifacts that existed as part of the original UCSD data sets (t-test_{(total vs detected)} p=0.870). No sub-threshold synthetic artifacts of any type were detected by our artifact detection software module (t-test_{(total vs detected)} p<0.0001) demonstrating the lack of false positive detection events. However, threshold synthetic artifacts were detected with nearly 100% accuracy (t-test_{(total vs detected)} p=0.495). Overall, 1382 events were detected of 1344 pre-existing and synthetic artifacts with a false detection rate of 2.8 percent. An example can be seen in FIG. 8A and the summary of this analysis is provided in FIG. 8B.

### Example 7

### Verification and Validation of the Spectral Sub-Band Analysis Software

The spectral analysis module was designed to accept cleaned data from the artifact detection software module, window the data with a Bartlet windowing function, and then spectrally transform the data using the MATLAB FFT( ) function. In addition to these standardized analysis routines, the spectral analysis module permitted the user to select other windowing functions (i.e. Hann, Hamming, etc.) as well as other spectral estimation techniques, including multi-taper spectral estimation using Slepian sequences, to minimize spectral leakage.

Furthermore, the spectral analysis module automatically generated Power Spectral Density (PSD) plots from recorded EEG data as well as summary Comma-Separated Value (CSV) files of the spectral analysis results. The PSD plots were additionally sent to the Microsoft PowerPoint program for further report generation automatically by the spectral analysis module. Summary CSV files provide a general data format for the spectral analysis results that can be further analyzed in JMP (statistics package from SAS) or used for more complex scientific graphing in KaleidaGraph (purchased from Synergy Software).

An additional software analysis module was created to generate FFT spectral sub-band metrics as a part of our signal analysis suite. This module has the ability to generate sub-band metrics from the spectral analysis module output that include:
i) Spectral power within each δ, θ, α, and β EEG frequency sub-bands;
ii) Arithmetic and Geometric means of each sub-band for the eyes-closed and eyes-open conditions; and
iii) Ratios of Arithmetic and Geometric means of each sub-band for the eyes-closed and eyes-open conditions.
   The spectral sub-band metric module automatically generated plots of the Arithmetic and Geometric means in addition to ratios of those means. Results from this analysis were plotted and sent to Microsoft PowerPoint for further report generation as well as written to CSV files for further analysis.

Testing and validation of the spectral analysis module was completed as follows. Timestamp data was extracted from the UCSD data files CBA1ff01, down-sampled to 128 Hz, and formatted for use. This timestamp array was used to generate seven synthetic analog signals. These seven in silico signals are illustrated in FIG. 9 and included:
1) White Noise with a Gaussian distribution (mean=0 mV, StDev=0.1 mV);
2) Cosine wave at 2.0 Hz (Delta Band; mean=0 mV, StDev=1 mV)+White Noise (from 1);
3) Cosine wave at 5.5 Hz (Theta Band; mean=0 mV, StDev=1 mV)+White Noise (from 1)+Delta;
4) Cosine wave at 10 Hz (Alpha Band; mean=0 mV, StDev=1 mV)+White Noise (from 1)+Delta+Theta;
5) Cosine wave at 21 Hz (Beta Band; mean=0 mV, StDev=1 mV)+White Noise (from 1)+Delta+Theta+Alpha;
6) Cosine wave at 40 Hz (Gamma Band; mean=0 mV, StDev=1 mV)+White Noise (from 1)+Delta+Theta+Alpha+Beta; and
7) Fractional summation of White Noise and all cosine waves that included 0.1*White Noise+0.25*Delta+0.33*Theta+0.5*Alpha+0.1*Beta.

The spectral analysis module was tested by running the spectral analysis code against each of these traces. Spectrograms, illustrating the evolution of the power spectrum over time, and power spectra of the entire files were generated (FIG. 9A, 9B, 9C). The spectral analysis module successfully identified the spectral power of each frequency contained in each data trace. Spectral leakage was nominal (0.25 Hz), such that shoulder frequency bins (e.g. 9.75, and 10.25 Hz bins surrounding the 10 Hz bin) contained a very small portion of the spectral power generated by the 10 Hz cosine waveform. Calculations were identical between spectrogram time frames as well as across runs. Attenuated input waveforms (seventh synthetic trace) were appropriately calculated as fractional relative power measurements across frequencies and sub-band quantifications (see FIG. 9D).

### Example 8

### Alzheimer's Disease Pilot Study Recruitment and Clinical Protocol

A clinical protocol was written and approved by an independent Institutional Review Board. Subjects were enrolled based on Mini-Mental State Exam performance into either an Alzheimer's disease group (with MMSE less than 28 but greater than 20) or healthy normals enrolled to a Control (CTL) group. A total of N=14 CTLs and N=10 AD subjects were enrolled with the demographics of 13 CTLs shown in FIG. 10. History of seizure or epilepsy was among the exclusion criteria the lead neurologist established.

### Example 9

### Collection of Time Series EEG Data

The study coordinator established Informed Consent with each subject according to the IRB approved clinical protocol. Moreover, she collected anywhere from 10 to 18 blocks of EEG data according to the task protocol shown in FIG. 11. An example time series EEG trace is shown in FIG. 12 covering a two-second period. Traces were sampled at 128 sam/sec with a 10-bit ADC in a NeuroSky MindSet Pro headset coupled via Bluetooth to a Dell Inspiron 1545 laptop PC using NeuroView software.

### Example 10

### Pre-Processing Artifact Detection Followed by Fourier Spectral Signal Analysis to Produce a Summary Feature Table for Each Subject for Each Block Task of Data

Each block of EEG data was pre-processed according to the system and methods of the present invention and then spectrally transformed and time averaged with a sliding 8 sec (1024 sample) window to produce a time averaged PSD like the one shown in FIG. 13. All signal analysis was conducted blinded to subject clinical disease diagnosis so as to remove any chance for bias. The feature extracted data table had roughly 120 variables.

### Example 11

### Univariate Statistical Analysis and Predictive Model Building for Classifiers of Disease State (AD vs CTL) in the Pilot Study Data

The blinded table of extracted features or markers was passed from the signal analyst to the statistician for uni-variate statistical analysis. Using JMP 8.0 software, each of the roughly 120 variables for each task for each subject was analyzed for statistical significance across the diagnostic group AD vs CTL. As shown in the literature, the AD brain exhibits a spectral slowing relative to CTL subjects. As shown in FIG. 14A, the lower frequency theta sub-band in AD subjects exhibits an elevation relative to CTL subjects. Complementary to this observation, one observes the relative beta sub-band suppressed with less power in AD subjects compared to CTL (FIG. 14B) as the power has shifted to the slower theta sub-band. Also consistent with the literature, the overall mean frequency of the relative PSD has shifted from an average of just above 11 Hz in CTL subjects to around 8 Hz on average in the AD subjects. T-Test False Positive Rate p-values are calculated and shown as insets for each graph.

Interesting, new signatures or classifiers have begun to emerge from the uni-variate analysis to include the relative power in the 18-20 Hz band. This marker provides preliminary excellent diagnostic performance, where a nominal logistical regression in JMP 8.0 determined that a cut point around 0.27 would optimally dichotomize the diagnostic group designation. ROC curve analysis was conducted to derive sensitivity and specificity of 85%190%, with PPV of 92% and NPV of 82% respectively. This can be seen in the 2×2 diagnostic table of FIG. 16.

### Example 12

### Prophetic: Conduct Multi-Variate Predictive Model Building to Find Better Composite Classifiers

Moreover, using established multi-variate predictive statistical methods, one can conduct multi-variate statistical analysis to build predictive statistical models that include from 2 to 10 variables from among the various tasks and features extracted in a given clinical protocol. It is well known that linear discriminant analysis, random forest, shrunken-centroids and other multi-variate approaches to construct composite signatures that classify subjects could be used on the summary feature data table in addition the uni-variate signatures and analysis conducted.

### Example 13

### Prophetic: Conduct Sports Concussion or mTBI Protocol Consisting of Cognitive Assessment, Vestibular/Balance, Auditory, and Visual Stimulation

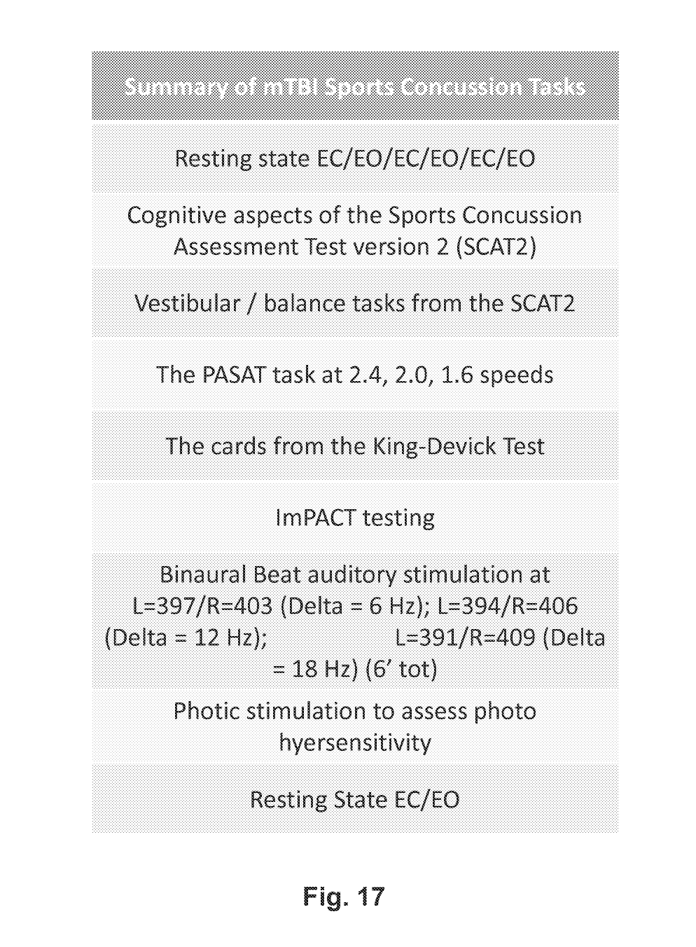

Alternatively, one could tailor a brain assessment battery towards sports concussion diagnosis and monitoring by combining simultaneous EEG recording with various tasks focused on sports concussion and mild traumatic brain injury. An prophetic example of such a battery can be seen in FIG. 17 where a subject would undergo resting state EC and EO conditions, cognitive elements of the SCAT2, vestibular and balance tasks from the SCAT2, the PASAT task, the King-Devick test, the ImPACT testing, binaural beats or auditory stimulation to assess tinnitus, photic stimulation to assess photo hypersensitivity and finally resting state EC and EO. It should be clear that not all tasks need be included and could simply just be a single task or a minimal combination of the statistically important ones. Moreover, elements from the Standard Concussion Assessment Test version 2 or SCAT2 could be used as tasks, including the Graded Symptom Checklist (GSC), Standard Assessment of Concussion (SAC), Balance Error Scoring System (BESS) or other symptom, cognitive, or vestibular tasks or challenges. Ideally, one would use passive tasks that do not require the cooperation of the subject.

### Example 14

### Feature Extraction with Discrete Wavelet Transformation (DWT) and Statistical Testing on the Alzheimer's Disease Pilot Study

### Discrete Wavelet Transform EEG Feature Extraction

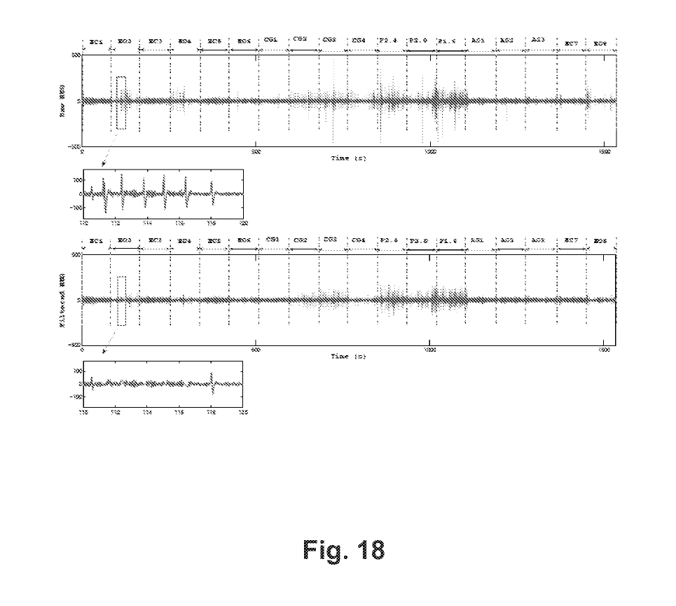

FIG. 18 is an example of a raw EEG signal of a subject (Subject 11) before (top) and after (bottom) artifact detection. Discrete Wavelet Transformation analyzes such a signal at different resolutions through its decomposition into several successive frequency bands by utilizing a scaling function φ(t) and a wavelet function ψ(t), associated with low-pass and high-pass filters, respectively. A useful property of these functions is that they can be obtained as a weighted sum of the scaled (dilated) and shifted versions of the scaling function itself:

$$\begin{matrix}φ ( t ) = {\sum }_{n} h [ n ] φ ( 2 t - n ) , & ( 1 ) \\ ψ ( t ) = {\sum }_{n} g [ n ] φ ( 2 t - n ) . & ( 2 )\end{matrix}$$

The coefficients (weights) h[n] and g[n] that satisfy (1) and (2) constitute the impulse responses of the low-pass and high-pass filters and define the type of the wavelet. The original EEG signal x(t) forms the discrete time signal x[n], which is first passed through a half-band high-pass filter (g[n]) and a low-pass filter (h[n]). Filtering followed by sub-sampling constitutes one level of decomposition and can be expressed as follows:

$$\begin{matrix}{d}_{1} [ k ] = {y}_{high} [ k ] = {\sum }_{n} x [ n ] \cdot g [ 2 k - n ] , & ( 3 ) \\ {α}_{1} [ k ] = {y}_{low} [ k ] = {\sum }_{n} x [ n ] \cdot h [ 2 k - n ] , & ( 4 )\end{matrix}$$

where d₁ and a₁ are level 1 detail and approximation coefficients, respectively, y_{high}[k] and y_{low}[k] are the outputs of the high-pass and low-pass filters after the sub-sampling.

DWT TABLE 3
DWT sub-band frequencies and the corresponding EEG frequency
bands. Sub band Frequency Range Corresponding EEG (Hz) frequency
band (Hz).

| | Frequency Range | Corresponding EEG |
|---|---|---|
| Sub band | (Hz) | frequency band (Hz) |
| D₁ | 30-60 | γ (>30) |
| D₂ | 15-30 | β (13-30) |
| D₃ | 7.5-15 | α (8-13) |
| D₄ | 3.75-7.5 | θ (4-8) |
| D₅ | 1.875-3.75 | δᵤ (2-4) |
| A₅ | 1-1.875 | δₗ (0-2) |

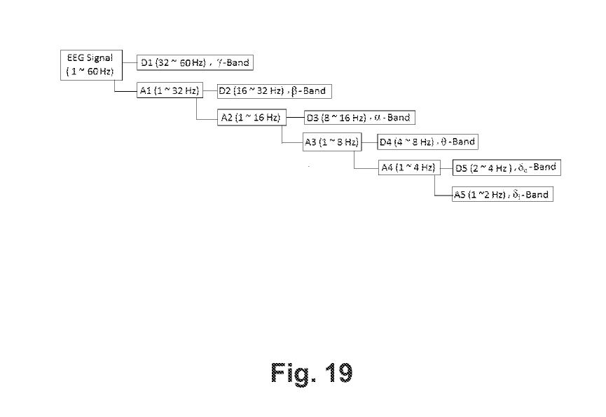

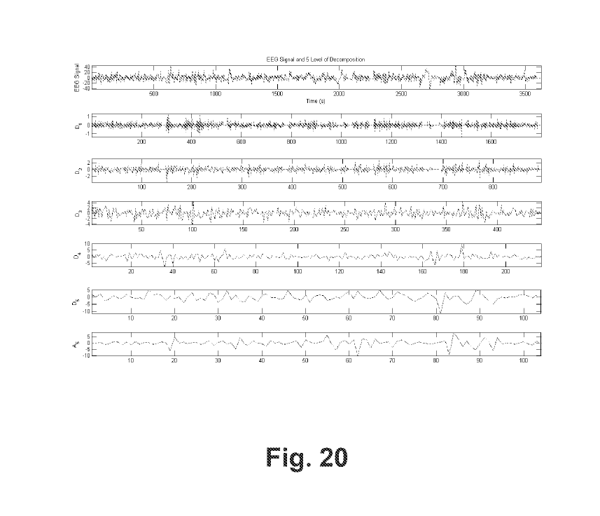

This procedure, called sub-band coding, is repeated for further decomposition as many times as desired or until no more sub-sampling is possible. At each level, it results in half the time resolution (due to sub-sampling) and double the frequency resolution (due to filtering), allowing the signal to be analyzed at different frequency ranges with different resolutions. FIG. 19 is a diagram showing the discrete wavelet transformation decomposition scheme with 5 levels of decomposition, where D₁-D₅ and A₅ represent the signal. In this analysis, we went through five levels of decompositions resulting in D₁ (approximately related to γ frequency band) through D₅ (approximately related to upper δ frequency band) and A₁ through A₅ (approximately related to lower δ frequency band). FIG. 20 shows these five levels of decomposition for the EEG signal. D₁ through D₅ sub-bands along with the A₅ sub-band consist the DWT representation of the EEG signal. DWT Table 3 shows these sub-bands with their frequency ranges and their corresponding EEG major frequency bands. However, not all these sub-bands are useful and reliable. Since the effective sampling rate of our EEG recording device is fₛ=125 Hz, we considered frequencies above 30 Hz (approximated half of Nyquist) unreliable. Hence, D₁ sub-band (γ frequency band) was not used in the subsequent analysis. Moreover, the analog filters employed in the headset had a cutoff of approximately 1 to 2 Hz. Those filters have undisclosed properties making the signal in 0-2 Hz unreliable. Hence, A₅ sub-band (lower 8 frequency band) features were also removed from the analysis. As a result, the effective sub-bands used in this study were D₂-D₅.

Having the sub-bands of EEG signals, we can extract the common statistical features from DWT analysis. In this study, we selected the minimum, maximum, standard deviation (STD), skewness and kurtosis values as well as average power of the wavelet coefficients as the candidate statistical features. These values were computed at each level of DWT decomposition separately for each recording state of the subjects. Note that, we did not consider the mean values since we had subtracted the mean before processing the data. DWT Table 4 lists all extracted features for the sub-bands and those selected for the analysis.

Choosing a suitable mother wavelet function is the most important factor in a reliable wavelet transform analysis. Daubechies family of mother wavelets, especially Daubechies2 (db2), has been reported to have a better accuracy compared to most other mother wavelet functions. Hence, we started our analysis with db2 wavelet and then moved on to four other wavelets from the Daubechies family (db4-db10) to extract EEG features. The number of statistically significant EEG features of AD patients compared to Control subjects, identified by the five different wavelets, are shown in DWT Table 5. It can be seen that db6 and db8 yield more features and are thus are the best choices for mother wavelet functions.

DWT TABLE 4
Statistical features of all the sub-bands and the statistical features under
study.

| | All Frequency Bands | Selected frequency Bands |
|---|---|---|
| Mean | D₁-D₅, A₅ | |
| Power Mean | D₁-D₅, A₅ | D₂-D₅ |
| Minimum | D₁-D₅, A₅ | D₂-D₅ |
| Maximum | D₁-D₅, A₅ | D₂-D₅ |
| STD | D₁-D₅, A₅ | D₂-D₅ |
| Skewness | D₁-D₅, A₅ | D₂-D₅ |
| Kurtosis | D₁-D₅, A₅ | D₂-D₅ |

DWT TABLE 5
Number of features derived by different Daubechies family of wavelets.

| Mother Wavelet | # of Significant features |
|---|---|
| Daubechies2 (db2) | 30 |
| Daubechies4 (db4) | 33 |
| Daubechies6 (db6) | 43 |
| Daubechies8 (db8) | 47 |
| Daubechies10 (db10) | 39 |

However, we used db6 due to more reliable statistically significant features as indicated by lower p-values (not listed here). As an example, DWT FIG. 20 shows the EEG signal from subject 22 during the One Card Learning recording task after each level of decomposition by db6 wavelet function.

### Discriminating Features: Statistical Testing

Initially, a two-tailed t-test was employed, a simple and common statistical testing method, to compare the signals from 10 AD patients with the 14 Control (CN) subjects. However, a t-test requires normal distribution of data which was not a valid assumption for some of the data in the AD pilot study. Therefore, the Kruskal-Wallis test, a non-parametric test based on Chi-squared distribution, was utilized to improve the suitability of the approach. The Kruskal-Wallis one-way analysis of variance by ranks is a method for testing whether samples originate from the same distribution. Since it is a non-parametric method, the Kruskal-Wallis test does not assume a normal distribution. This method has been used for comparing more than two samples that are independent, or not related. The parametric equivalence of the Kruskal-Wallis test is the one-way analysis of variance (ANOVA). The factual null hypothesis is that the populations from which the samples originate have the same median. When the Kruskal-Wallis test leads to significant results, then at least one of the samples is different from the other samples. The test statistics of Kruskal-Wallis is defined as:

$$K = \frac{12}{{n}_{T} ( {n}_{T} + 1 )} {\sum }_{i} \frac{{T}_{i}^{2}}{{n}_{i}} - 3 ( {n}_{T} + 1 ) \qquad ( 5 )$$

where n, is the number of observations from sample i (i=1, 2, . . . , k), n_{T} is the combined (total) sample size (n_{T}=Σ nᵢ) and Tᵢ denotes the sum of the ranks for the measurement in sample i after the combined sample measurements have been ranked. The test does not identify where the differences occur or how many differences actually occur.

The result of Kruskal-Wallis statistical testing method and the corresponding p-values related to the significant features are shown in DWT Table 6. According to this table, the second eyes-open state (EO4) yields the most number of statistically significant features followed by the third eyes-open state (EO6) and the third eyes-closed state (EC5). The min {D2} (β frequency band) of the One Card Learning cognitive task, maximum {D3} (α frequency band) of the One Card Back cognitive task, skewness {D3} (α frequency band) of the One Card Back cognitive task, and the skewness {D2} (β frequency band) of the auditory binaural beat stimulation at Δ=6 Hz were the statistically significant features in the activated states.

DWT TABLE 6
Statistically significant EEG features of AD based on Kruskal-Wallis test and their p-value.
Any task/feature combination with a value less than 0.05 is considered statistically significant
and thus a candidate EEG feature for use in statistical predictive models.

| | EC | EO | EC | EO | EC | EO | CG | CG | CG | CG | P | P | AS | AS | AS | EC | EO |
|---|---|---|---|---|---|---|---|---|---|---|---|---|---|---|---|---|---|
| | 1 | 2 | 3 | 4 | 5 | 6 | 1 | 2 | 3 | 4 | 2.4 | 2.0 | 1 | 2 | 3 | 7 | 8 |
| Power Mean | — | .04 | — | .062 | — | .035 | — | — | — | — | — | — | — | — | — | — | — |
| D₂ | | | | | | | | | | | | | | | | | |
| Power Mean | — | — | — | .022 | .04 | — | — | — | — | — | — | — | — | — | — | — | — |
| D₃ | | | | | | | | | | | | | | | | | |
| Power Mean | — | .03 | — | .0001 | .001 | — | — | — | — | — | — | — | — | — | — | — | .026 |
| D₄ | | | | | | | | | | | | | | | | | |
| Power Mean | — | — | — | .0005 | — | .046 | — | — | — | — | — | — | — | — | — | — | — |
| D₅ | | | | | | | | | | | | | | | | | |
| Minimum | — | .04 | — | .007 | — | — | — | — | .04 | — | — | — | — | — | — | — | — |
| D₂ | | | | | | | | | | | | | | | | | |
| Minimum | — | — | — | .011 | .001 | — | — | — | — | — | — | — | — | — | — | — | — |
| D₃ | | | | | | | | | | | | | | | | | |
| Minimum | — | — | — | — | .007 | .022 | — | — | — | — | — | — | — | — | — | — | — |
| D₄ | | | | | | | | | | | | | | | | | |
| Minimum | — | — | — | .002 | — | .016 | — | — | — | — | — | — | — | — | — | — | — |
| D₅ | | | | | | | | | | | | | | | | | |
| Maximum | — | — | — | .016 | — | .016 | — | — | — | — | — | — | — | — | — | — | — |
| D₂ | | | | | | | | | | | | | | | | | |
| Maximum | — | — | — | — | — | — | — | — | — | .0098 | — | — | — | — | — | — | — |
| D₃ | | | | | | | | | | | | | | | | | |
| Maximum | — | — | — | .026 | .021 | — | — | — | — | — | — | — | — | — | — | — | — |
| D₄ | | | | | | | | | | | | | | | | | |
| Maximum | — | — | — | .04 | — | .016 | — | — | — | — | — | — | — | — | — | — | — |
| D₅ | | | | | | | | | | | | | | | | | |
| STD | — | .04 | — | .003 | — | .035 | — | — | — | — | — | — | — | — | — | — | — |
| D₂ | | | | | | | | | | | | | | | | | |
| STD | — | — | — | .022 | .047 | — | — | — | — | — | — | — | — | — | — | — | — |
| D₃ | | | | | | | | | | | | | | | | | |
| STD | — | .022 | — | .0001 | .001 | — | — | — | — | — | — | — | — | — | — | — | .026 |
| D₄ | | | | | | | | | | | | | | | | | |
| STD | — | — | — | .0006 | — | .04 | — | — | — | — | — | — | — | — | — | — | — |
| D₅ | | | | | | | | | | | | | | | | | |
| Skewness | — | — | — | — | — | — | — | — | — | — | — | — | .0 [symbol] 1 | — | — | — | — |
| D₂ | | | | | | | | | | | | | | | | | |
| Skewness | — | — | — | — | — | — | — | — | — | .032 | — | — | — | — | — | — | — |
| D₃ | | | | | | | | | | | | | | | | | |
| Skewness | — | — | — | — | — | — | — | — | — | — | — | — | — | — | — | — | — |
| D₄ | | | | | | | | | | | | | | | | | |
| Skewness | — | — | — | — | — | — | — | — | — | — | — | — | — | — | — | .0005 | — |
| D₅ | | | | | | | | | | | | | | | | | |
| Kurtosis | — | — | — | — | — | — | — | — | — | — | — | — | — | — | — | — | — |
| D₂ | | | | | | | | | | | | | | | | | |
| Kurtosis | — | — | — | — | — | — | — | — | — | — | — | — | — | — | — | — | — |
| D₃ | | | | | | | | | | | | | | | | | |
| Kurtosis | — | — | — | — | — | — | — | — | — | — | — | — | — | — | — | — | — |
| D₄ | | | | | | | | | | | | | | | | | |
| Kurtosis | — | — | — | — | — | .0 [symbol] | — | — | — | — | — | — | — | — | — | — | .02 |
| D₅ | | | | | | | | | | | | | | | | | |

[symbol]

indicates data missing or illegible when filed

### Decision Tree

Since several significant features were identified in our study from Kruskal-Wallis statistical method, we choose to investigate further using an algorithm to determine the most dominant and reliable discriminating feature of AD patients. Therefore, we applied a widely used classification method or predictive statistical model called the decision tree analysis. Decision tree analysis holds several advantages over traditional supervised methods, such as maximum likelihood classification. It does not depend on assumptions of distributions of the data and therefore is a non-parametric method. Another valuable advantage of decision tree is its ability to handle missing values, which is a very common problem in dealing with the biomedical data.

A tree T is made up of nodes and branches. A node t is designated as either an internal or a terminal node. Internal nodes can split into two children (t_{L} for the left branch and t_{R} for the right branch) while the terminal nodes cannot. The most important aspect of a decision tree induction strategy is the split criteria, which is the method of selecting an attribute test that determines the distribution of training objects into sub-sets upon which sub-trees are built consequently.

In this study, we used two well-known split criteria: Gini and Twoing index. Each of the splitting rules attempts to segregate data using different approaches. The Gini index is defined as:

$$Gini ( t ) = {\sum }_{i} {p}_{i} ( 1 - {p}_{i} ) \qquad ( 6 )$$

where pᵢ is the relative frequency of class i at node t, and node t represent any node at which a given split of the data is performed. pᵢ is determined by dividing the total number of observations of the class by the total number of observations. The Twoing index is defined as:

$$Twaing ( t ) = \frac{{p}_{L} {p}_{R}}{4} {( {\sum }_{i} ( \vert p ( i \vert {t}_{L} ) - p ( i \vert {t}_{R} ) \vert ) )}^{2} \qquad ( 7 )$$

where L and R refer to the left and right sides of a given split respectively, and p(i|t) is the relative frequency of class i at node t.

Initially, we applied the decision tree with Twoing index to the resting state (EC1-EO6) extracted EEG features, as shown in FIG. 21. The algorithm identified the standard deviation {D4} (θ frequency band, 4-8 Hz) of the second eyes-open state (EO4), as the first and most dominant discriminating feature of AD patients. The second discriminating feature was the power mean {D2} (β frequency band, 13-30 Hz) of the second eyes-open state (EO4). These results indicate that if the standard deviation value of the {D4}, corresponding to θ frequency band, of EO4 state of a subject is greater than 2.053, then that subject is identified as an AD patient. Otherwise, if the {D2} power mean value, corresponding to the β band, in the EO4 state is less than 0.158 (following the red line in decision tree), then the subject is again identified as an AD patient. These discriminating features were also determined to be statistically significant by Kruskal-Wallis testing method, as shown in DWT Table 6.

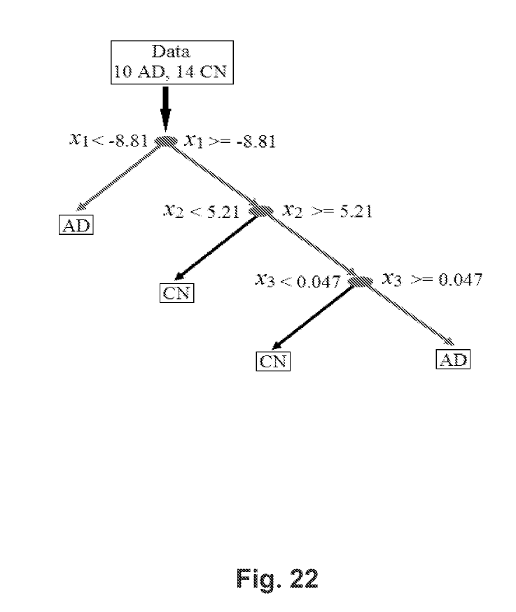

Next, we applied the decision tree algorithm with Twoing index to active state recordings only with the result shown in FIG. 22. According to these results, min {D4} (θ frequency band, 4-8 Hz) of the auditory binaural beat stimulations with Δ=12 Hz (AS2) was the first and most dominant discriminating feature of AD patients. The max {D3} (α frequency band, 8-13 Hz) of the auditory binaural beat stimulations at Δ=6 Hz (AS1) was the next feature followed by the skewness {D3} (α frequency band, 8-13 Hz) of the One Card Back cognitive task (CG4). These results indicate that if the minimum value of the {D4}, corresponding to the θ frequency band, during the binaural beat auditory stimulations at Δ=12 Hz of a subject is less than −8.81, then that subject is identified as an AD patient. Otherwise, if the {D3} band maximum value, corresponding to the α frequency band, during binaural beat auditory stimulations at Δ=6 Hz of the subject is greater than or equal to 5.21 and the skewness {D3} distribution, corresponding to the α band, during the One Card Back cognitive task of the subject is greater than or equal to 0.047, then the subject is identified as an AD patient. The last discriminating feature was also determined to be statistically significant by Kruskal-Wallis testing method, as shown in DWT Table 6.

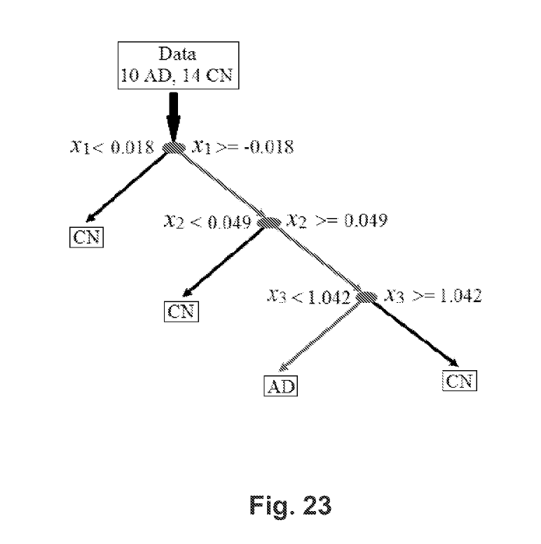

Combining all recording states together, we applied the decision tree algorithm to all features, as shown in FIG. 23. We used the Gini index in this case because the Twoing index did not yield a convincing result. The algorithm identified the skewness {D5} (upper δ frequency band, 2-4 Hz) of fourth Eyes-Closed state (EC7) as the first and most dominant discriminating feature of AD patients. The next feature was the power mean {D2} (β frequency band, 13-30 Hz) of the PASAT with the 2.0 (s) intervals task followed by the power mean {D2} (β frequency band, 13-30 Hz) of the first eyes-open (EO2) state. These results indicate that if the distribution of δ frequency band during EC7 of has skewness greater than or equal to −0.018 and the power mean value of β frequency band of PASAT 2.0 (s) interval of the subject is also greater than or equal to 0.049 and the mean power value of the β frequency band of the EO2 of that subject is less than 1.042, then the subject is identified as an AD patient. The first and third of these features were also determined to be statistically significant by Kruskal-Wallis testing method, as shown in DWT Table 6. It should be noted that any task/feature combination that appears in the Decision Tree analysis or DWT Table 6 are candidate EEG features to be used in predictive statistical models.

### Example 15

### Feature Extraction with Continuous Wavelet Transformation (CWT) and Statistical Testing on the Alzheimer's Pilot Study

### Continuous Wavelet Transformation EEG Feature Extraction

If x(t) is a square integrable function of time, t, then its CWT is defined as:

$$C ( a , b ) = \frac{1}{\sqrt{a}} {\int }_{- ∞}^{+ ∞} x ( t ) {ψ}^{*} ( \frac{t - b}{a} ) d t , \qquad ( 1 )$$

where a, b ∈ R, a≠0, and R is the set of real numbers, a is the dilation parameter called ‘scale’ and b is the location parameter of the wavelet, y(t) is the wavelet function called the “mother wavelet”, superscript “*” denotes the complex conjugate of the function, and 1 √a is used to normalize the energy such that it stays at the same level for different values of a and b.

In this study, a commonly used complex-valued wavelet Morlet function was selected:

$$ψ ( t ) = {π}^{- \frac{1}{4}} {e}^{{iω}_{0} t} {e}^{- \frac{{t}^{2}}{2}} , \qquad ( 2 )$$

where y(t) is the wavelet function that depends on a non-dimensional time parameter t, and i denotes the imaginary unit. This wavelet function forms two exponential functions modulating a Gaussian envelope of unit width, where the parameter w₀ is the non-dimensional frequency parameter, here taken to be 5 to satisfy the admissibility condition and have a zero average. The relationship between CWT scales and frequency is roughly of inverse form such that low scale corresponds to high frequency and vice versa. The Wavelet Toolbox of MATLAB (the MathWorks) uses the following formula to map between a scale and a pseudo-frequency:

$${F}_{a} = \frac{{F}_{c}}{a \cdot Δ} , \qquad ( 3 )$$

where a is a CWT scale, Δ is the sampling period (1 fs), F_{c} is the center frequency of the wavelet function (0.8125 Hz for Morlet), and Fₐ is the pseudo-frequency corresponding to scale a and given as:

$${F}_{a} = \frac{104}{a} . \qquad ( 4 )$$

CWT TABLE 7
Ranges for Wavelet Scales and Their Corresponding Pseudo-
Frequency and Brain Bands.

| Scale | Pseudo-Frequency | Corresponding |
|---|---|---|
| Range | Range (Hz) | EEG Band |
| [1.5-3.5] | [30-60] | γ |
| [3.5-5] | [20-30] | β_{U} |
| [5-8.5] | [13-20] | β_{L} |
| [3.5-8.5] | [13-30] | β |
| [8.5-10] | [10-13] | α_{U} |
| [10-13] | [8-10] | α_{L} |
| [8.5-13] | [8-13] | α |
| [13-18] | [6-8] | θ_{U} |
| [18-26] | [4-6] | θ_{L} |
| [13-26] | [4-8] | θ |
| [26-40] | [2-4] | δ_{U} |
| [26-80] | [1-4] | δ |

We calculated the coefficients of CWT from Eq. (1) for the scale range of [1.5-80] with a scale-step of 0.1 for all subjects in the pilot Alzheimer's study. Next, we computed the geometric mean power spectrum of the wavelet coefficients of each phase:

$${P}_{j} = \frac{1}{n} {\sum }_{i = 0}^{n - 1} {\vert {x}_{i} \vert}^{2} , j = 1 , … , 786 , \qquad ( 5 )$$

where xᵢ's are the computed coefficients of the signal at each scale and 786 is the total number of scales. The powers are then averaged over time through the calculation of their geometric means.

In this study, we analyzed the major brain frequency bands, δ, θ, α, β, and γ and their upper and lower ranges. CWT Table 7 shows different scale ranges and their corresponding pseudo-frequency range, according to Eq. (4), and corresponding EEG major frequency bands. Hence, the mean value of geometric means at each scale range gives us their corresponding absolute power, P_{band}. We also calculated the relative powers within each scale range normalized based on the scale range's total power.

### Statistical Testing

We initially used a two-tailed t-test to compare the signals from AD patients with those of controls and determine the statistically significant discriminant EEG features. However, t-test requires normal distribution of data which was not always a valid assumption in our study. Hence, we used the Kruskal-Wallis method, a non-parametric statistical test based on Chi-squared distribution, to ensure reliability. Both Kruskal-Wallis and t-test determined similar statistically significant features. There were, however, some additional features identified by t-test which were deemed unreliable and discarded. The results of the Kruskal-Wallis testing method are shown in CWT Table 8 with the corresponding false positive rate p-values. These results represent the statistically significant discriminating features of AD patients under sequential resting eyes-closed and eyes-open states. The results show that the highest number of statistically significant features for both relative and absolute powers are observed in the second eyes-open state (EO4). The second highest number of statistically significant features are observed during the third eyes closed (EC5) and eyes-open (EO6), while there are very few statistically significant features during EC1 through EC3. Note that, since βₗ and βᵤ of both relative and absolute powers in EO4 and EO6 are statistically significant features, the full β band relative and absolute mean powers are also significant features. These features indicate that the relative and absolute mean β powers are significantly lower for AD patients when compared to control subjects. Similarly, the θ band absolute powers in EO4 and EC5 states demonstrate statistically significant features at both lower and upper θ ranges. In this case, the features are significantly higher for AD patients when compared to control subjects. Note that, these results are consistent with other reported FFT results in the literature.

### Decision Tree

There are many features identified, as shown in CWT Table 8 which require further validation through more clinical studies and data collection. Hence, an algorithm to determine and classify the most reliable features identifying AD patients is desirable for the current study. We applied a decision tree algorithm to determine the most significant and dominant discriminating feature of AD patients. The tree is made up of nodes and branches. A node t is designated as either an internal or a terminal node. Internal nodes can split into two children while the terminal nodes cannot. Unlike the statistical testing methods, which use data distribution for comparison of different groups, decision tree attempts to segregate data using different splitting criteria. In this study, we used a well-known splitting criterion called the Gini index which is defined as:

$$Gini ( t ) = {\sum }_{i} {p}_{i} ( 1 - {p}_{i} ) \qquad ( 6 )$$

where pᵢ is the relative frequency of class i at node t, and node t represent any node at which a given split of the data is performed. pᵢ is determined by dividing the total number of observations of the class by the total number of observations.

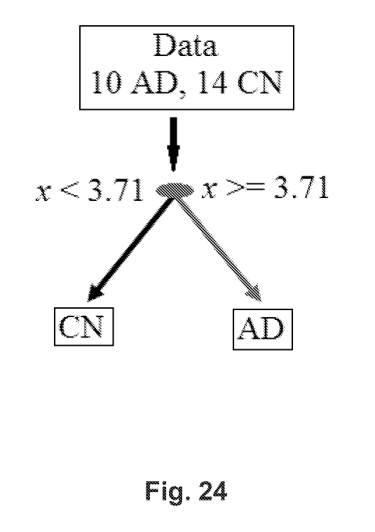

The top line result of the decision tree algorithm for comparing the AD and control subjects in this study is shown in FIG. 24. The algorithm clearly indicates (with 100% confidence) that absolute power of CWT coefficients in the scale range corresponding to the θ major brain frequency band (4-8 Hz) of the second eyes-open state (EO4) in the sequential EEG recordings is the most significant discriminating feature and the best identifier of AD patients. This implies that if the absolute power of CWT coefficients of the θ frequency band from the EO4 of a subject is greater than 3.71, in arbitrary units, then the subject is identified as an AD subject. This result shows that the absolute θ band mean power is significantly higher for AD patients when compared to control subjects and is consistent with the reported results in the literature. Note that, this feature was also determined to be statistically significant by Kruskal-Wallis and t-test statistical testing methods.

CWT TABLE 8
Significant Discriminating Features Based on Kruskal-Wallis Results.

| State | δᵤ | θₗ | θᵤ | αₗ | αᵤ | βₗ | βᵤ |
|---|---|---|---|---|---|---|---|
| | Relative Powers | | | | | | |
| EC1 | 0 | 0 | 0 | 0 | 0 | 0 | 0 |
| EO2 | 0 | 0 | 0 | 0 | 0 | 1, p = 0.046 | 1, p = 0.046 |
| EC3 | 0 | 0 | 0 | 0 | 0 | 0 | 0 |
| EO4 | 1, p = 0.0006 | 1, p = 8.7 × 10⁻⁵ | 0 | 0 | 1, p = 0.007 | 1, p = 0.004 | 1, p = 0.004 |
| EC5 | 0 | 1, p = 0.084 | 0 | 0 | 1, p = 0.025 | 1, p = 0.025 | 0 |
| EO6 | 0 | 1, p = 0.026 | 0 | 0 | 0 | 1, p = 0.035 | 1, p = 0.022 |
| | Absolute Powers | | | | | | |
| EC1 | 0 | 0 | 0 | 0 | 0 | 0 | 0 |
| EO2 | 0 | 0 | 1, p = 0.019 | 0 | 0 | 0 | 0 |
| EC3 | 0 | 0 | 1, p = 0.019 | 0 | 0 | 0 | 0 |
| EO4 | 1, p = 0.0005 | 1, p = 5.3 × 10⁻⁵ | 1, p = 0.007 | 0 | 1, p = 0.026 | 1, p = 0.004 | 1, p = 0.005 |
| EC5 | 0 | 1, p = 0.047 | 1, p = 0.029 | 0 | 1, p = 0.015 | 1, p = 0.018 | 0 |
| EO6 | 1, p = 0.040 | 1, p = 0.016 | 0 | 0 | 0 | 1, p = 0.046 | 1, p = 0.019 |

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

We claim:

1. A system for calibrating and/or verifying system performance of a remote portable EEG system having at least one EEG sensor, comprising:
at least one ground electrode;
a signal generator producing at least one channel of reference signals;
a wired cable assembly that connects the signal generator output to at least one EEG sensor and ground electrode; and
a programmed processor that generates test reference signals and collects responses generated by the EEG sensor to the test reference signals to confirm system calibration and and/or verify system performance of the remote portable EEG system.

2. The system of claim 1 wherein the signal generator includes a sound card assembled into a microprocessor based device.

3. The system of claim 1 wherein the reference signals generated include linear combinations of sine, square, and triangle waves of varying frequency and amplitude.

4. The system of claim 1 wherein the reference signals generated include a short circuit between the reference signal and ground enabling a short circuit noise assessment.

5. The system of claim 1 wherein the programmed processor is programmed with software algorithms that enable the coordination of the generation of reference signals and the data collection of such reference signals for automated system verification and validation.

6. The system of claim 1 wherein the wired cable assembly contains a voltage divider to diminish test reference signal amplitudes to physiologically relevant levels.

7. The system of claim 1 wherein the wired cable assembly contains a removable voltage divider to diminish test reference signal amplitudes to physiological levels when in place or to calibrate reference signal amplitudes on an individual device by device level when removed from the wired cable assembly.

8. A system for assessing the state or function of a subject's brain, comprising:
a portable EEG sensing device that acquires a subject's EEG signal data during cognitive or sensory testing; and
a feature extraction system that processes the subject's EEG signal data to establish a noninvasive biomarker in the brain that enables the classification, prognosis, diagnosis, monitoring of treatment, or response to therapy applied to the brain by measuring an extracted EEG feature or EEG features from a measured EEG signal when conducting a predetermined cognitive or sensory task, feature extraction system further measuring changes in the extracted EEG feature or EEG features over time, among multiple states, or compared to a normative database.

9. The system of claim 8 wherein the feature extraction system establishes a biomarker by assessing each block of EEG signal data from the subject to create a list of features, variables or metrics extracted from each block of EEG signal data collected during an individual cognitive task, said list of features, variables or metrics including at least one of: relative and absolute delta, theta, alpha, beta and gamma sub-bands, the theta/beta ratio, the delta/alpha ratio, the (theta+delta)/(alpha+beta) ratio, the relative power in a sliding two Hz window starting at 4 Hz and going to 60 Hz, the 1-2.5 Hz power, the 2.5-4 Hz power, the peak or mode frequency in the power spectral density distribution, the median frequency in the power spectral density, the mean or average (1^{st} moment) frequency of the power spectral density, the standard deviation of the mean frequency (square root of the variance or 2^{nd} moment of the distribution), the skewness or 3^{rd} moment of the power spectral density, and the kurtosis or 4^{th} moment of the power spectral density.

10. The system of claim 8 wherein the EEG feature or EEG features extracted by the feature extraction system includes the relative power spectral density within the 18<=f<=20 Hz frequency range of a measured EEG signal when conducting the predetermined cognitive or sensory task, said feature extraction system further establishing a cut-point between 0 and 100 percent for the relative power spectral density across the 18-20 Hz range.

11. The system of claim 8, wherein the non-invasive biomarker comprises statistically significant EEG features of Alzheimer's Disease based on the p-value of a statistical significance test applied to the subject.

12. The system of claim 8 wherein the predetermined cognitive or sensory task is a resting state Eyes Open task or Eyes Closed task.

13. The system of claim 8 wherein the predetermined cognitive or sensory task includes at least one of a Fixation task, a CogState Attention task, a CogState Identification task, a CogState One Card Learning task, a CogState One Card Back task, a Paced Arithmetic Serial Auditory Task (PASAT), a King-Devick Opthalmologic task, a neuro-opthalmologic task, a monaural beat auditory stimulation task, a binaural beat auditory stimulation task, an isochronic tone auditory stimulation task, a photic stimulation task, an ImPACT task, a SCAT2 task, a BESS task, a vestibular eye tracking task, or a dynamic motor tracking task.

14. The system of claim 8, wherein the feature extraction system further diagnoses a disease state of a brain and nervous system of a subject by acquiring EEG signal data of the subject during a resting state task using said portable EEG sensing device, measuring the relative power spectral density of the subject's EEG signal data in a designated frequency sub-band, applying a predetermined cut-point to dichotomize the power spectral density results into one or more biomarker states or classes, and determining which biomarker class a subject belongs to based on the subject's individual power spectral density measurement relative to the predetermined cut-point.

15. The system of claim 8, wherein the feature extraction system extracts an EEG feature or EEG features by applying discrete or continuous wavelet transformation analysis to the subject's EEG signal data to identify statistically meaningful features.
